(19) United States
(12) Patent Application Publication
(10) Pub. No.: US 2018/0338713 A1
(43) Pub. Date: Nov. 29, 2018

(54) INTEGRATED FLUIDIC CHIP FOR TRANSDERMAL SENSING OF PHYSIOLOGICAL MARKERS

(71) Applicant: Sandia Corporation, Albuquerque, NM (US)

(72) Inventors: Ronen Polsky, Albuquerque, NM (US); Philip Rocco Miller, Albuquerque, NM (US); Matthew W. Moorman, Albuquerque, NM (US); Susan M. Brozik, Albuquerque, NM (US); Ronald P. Manginell, Albuquerque, NM (US); David R. Wheeler, Albuquerque, NM (US); Thayne L. Edwards, Albuquerque, NM (US)

(21) Appl. No.: 14/538,096

(22) Filed: Nov. 11, 2014

Related U.S. Application Data
(60) Provisional application No. 61/902,617, filed on Nov. 11, 2013.

(51) Int. Cl. A61B 5/1473; A61B 5/1459; A61B 5/00; A61B 5/145; A61B 5/157; A61B 5/155; A61B 5/15; A61B 10/00

(52) U.S. Cl. CPC A61B 5/14735; A61B 5/1459; A61B 5/4839; A61B 5/14514; A61B 5/14517; A61B 5/14546; A61B 2010/008; A61B 5/155; A61B 5/150984; A61B 5/150229; A61B 10/0064; A61B 5/0004; A61B 5/157

(57) ABSTRACT

The present invention is directed to devices and methods for detecting one or more markers in a sample. In particular, such devices integrate a plurality of hollow needles configured to extract or obtain a fluid sample from a subject, as well as transducers to detect a marker of interest (e.g., an electrolyte). In some embodiments, the needles are provided as a disposable cartridge.

### CROSS-REFERENCE TO RELATED APPLICATION

This application claims the benefit of U.S. Provisional Application No. 61/902,617, filed Nov. 11, 2013, which is hereby incorporated by reference in its entirety.

### STATEMENT OF GOVERNMENT INTEREST

This invention was developed under Contract No. DE-AC04-94AL85000 between Sandia Corporation and the U.S. Department of Energy. The U.S. Government has certain rights in this invention.

### FIELD OF THE INVENTION

The invention relates to methods and apparatuses for electrochemical bioassays, and more particularly to miniaturizeable systems and their use for in vivo measurements.

### BACKGROUND

There is a need for the real-time monitoring of a human subject's physiological state without the constant presence of a healthcare professional. This need arises in various situations, including home care of disabled or elderly patients, remote monitoring of combatants in battlefield conditions, and remote monitoring of firefighters and other emergency response personnel during crisis deployment.

Furthermore, there is a need for a real-time platform that can monitor or detect possibly contagious or infectious diseases. In particular, the platform should require minimal handling by the healthcare professional, thereby reducing the chance of infection to the professional or further spread of the disease. If the platform requires a minimal sample from the subject (e.g., a minimal amount of blood or other biological fluids), then the amount of possibly contagious biohazard material is reduced. Other benefits include such platforms that are minimally invasive, thereby reducing discomfort or pain in patients.

One answer to these needs would be an autonomous remote diagnostic device that is capable of interfacing with the human subject and performing a variety of diagnostic functions directed to that subject. The field is open for the development of such devices.

### SUMMARY OF THE INVENTION

We have developed a microfluidic bioassay device that can be worn on an individual and can transdermally access a test sample (e.g., blood and/or interstitial fluid) to create a real-time long term autonomous diagnostic device to monitor physiological signatures. In alternative embodiments, the device includes a disposable cartridge configured to transdermally access the test sample. Such devices include a needle, lancet, or puncturing tool, etc. that is connected to a microfluidic chip that can extract blood and/or interstitial fluid. The extracted fluid is then run over downstream transducers (i.e., electrode arrays, optical sensors, etc.) for either direct monitoring of physiological markers or monitoring after first being subject to post-processing reactions.

In particular embodiments, the invention combines in vivo needle (e.g., microneedle) platforms with multifunctional lab-on-a-chip electrode arrays that are capable of detecting a diverse number of relevant biomarkers. In some instances, the invention incorporates various structures to provide an integrated device having multiple functionalities (e.g., extracting a test sample, delivering the test sample to an appropriate transducer or sensor, and/or detecting one or more markers). Accordingly, described herein are exemplary methods for fabricating needles and transducer arrays, designing disposable cartridges including such needles, optimizing such components (e.g., to detect a plurality of markers), and integrating such components in a packaged chip.

In one embodiment, microneedles are used as the puncturing tool. In vivo microneedles are known. They have been shown to be an effective and minimally invasive method for transdermal access for fluid exchange with living subjects. Microneedles are advantageous over conventional needles and lancets for some applications because they cause minimal discomfort. This is because microneedles do not interact with deeper layers of the dermis, which are associated with sensation and pain.

The most common use of microneedles has involved drug delivery applications. However, we have recognized that microneedles can also be used to extract fluid for the detection of physiological markers, such as glucose, lactate, and pH or any described herein.

Thus, we have developed an in vivo microneedle platform integrated with multifunctional lab-on chip electrode arrays that can detect various diagnostic biomarkers. The microneedles are effective for extracting interstitial fluid that is directed through fluidic channels to electrochemical transducers for monitoring.

However, the needles can also be larger with dimensions in the millimeter-scale (e.g., 0.5 mm, 1 mm, 1.5 mm, 2 mm, 2.5 mm, 3 mm or more) to go deeper into a subject and extract blood as well as interstitial fluid. An anesthetic (such as, e.g., hirudin) can be secreted or used to coat the needle to minimize discomfort.

Accordingly, the present invention features a device for detecting one or more markers (e.g., any described herein, such as a protein, a toxin, etc.) in a sample including a plurality of hollow needles, where each needle has an interior surface facing the hollow lumen and an exterior surface. The needle can also include a distal end of the exterior surface, where at least one needle includes a puncturing edge. In some embodiments, at least one needle has a length of more than about 0.5 mm or from about 0.1 mm to about 7 mm (e.g., from 0.1 mm to 0.5 mm, 0.1 mm to 1 mm, 0.1 mm to 1.5 mm, 0.1 mm to 2 mm, 0.1 mm to 2.5 mm, 0.1 mm to 3 mm, 0.1 mm to 3.5 mm, 0.1 mm to 4 mm, 0.1 mm to 4.5 mm, 0.1 mm to 5 mm, 0.2 mm to 0.5 mm, 0.2 mm to 1 mm, 0.2 mm to 1.5 mm, 0.2 mm to 2 mm, 0.2 mm to 2.5 mm, 0.2 mm to 3 mm, 0.2 mm to 3.5 mm, 0.2 mm to 4 mm, 0.2 mm to 4.5 mm, 0.2 mm to 5 mm, 0.2 mm to 7 mm, 0.3 mm to 0.5 mm, 0.3 mm to 1 mm, 0.3 mm to 1.5 mm, 0.3 mm to 2 mm, 0.3 mm to 2.5 mm, 0.3 mm to 3 mm, 0.3 mm to 3.5 mm, 0.3 mm to 4 mm, 0.3 mm to 4.5 mm, 0.3 mm to 5 mm, 0.3 mm to 7 mm, 0.5 mm to 1 mm, 0.5 mm to 1.5 mm, 0.5 mm to 2 mm, 0.5 mm to 2.5 mm, 0.5 mm to 3 mm, 0.5 mm to 3.5 mm, 0.5 mm to 4 mm, 0.5 mm to 4.5 mm, 0.5 mm to 5 mm, 0.5 mm to 7 mm, 0.7 mm to 1 mm, 0.7 mm to 1.5 mm, 0.7 mm to 2 mm, 0.7 mm to 2.5 mm, 0.7 mm to 3 mm, 0.7 mm to 3.5 mm, 0.7 mm to 4 mm, 0.7 mm to 4.5 mm, 0.7 mm to 5 mm, 0.7 mm to 7 mm, 1 mm to 1.5 mm, 1 mm to 2 mm, 1 mm to 2.5 mm, 1 mm to 3 mm, 1 mm to 3.5 mm, 1 mm to 4 mm, 1 mm to 4.5 mm, 1 mm to 5 mm, 1 mm to 7 mm, 1.5 mm to 2 mm, 1.5 mm to 2.5 mm, 1.5 mm to 3 mm, 1.5 mm to 3.5 mm, 1.5 mm to 4 mm, 1.5 mm to 4.5 mm, 1.5 mm to 5 mm, 1.5 mm to 7 mm, 3 mm to 3.5 mm, 3 mm to 4 mm, 3 mm to 4.5 mm, 3 mm to 5 mm, and 3 mm to 7 mm). In other embodiments, the plurality of microneedles is provided in an array (e.g., two, three, four, five, six, seven, eight, nine, ten, fifteen, twenty, or more needles in array).

In further embodiments, the device includes a substrate coupled (e.g., directly or indirectly coupled) to the plurality of hollow needles, where the substrate includes one or more inlets in fluidic communication with a proximal end of at least one needle; a first channel coupled to the substrate and in fluidic communication with at least one inlet of the substrate; and one or more sensing transducers (e.g., one or more electrodes, such as in an array) in fluidic communication with the first channel, where at least one sensing transducer is configured to detect one or more markers in the sample.

In some embodiments, the device includes one or more components to operate the sensing transducer (e.g., a power source, a data-processing circuit powered by the power source and electrically connected to the transducer (e.g., a counter electrode, a reference electrode, and at least one said working electrode) and/or a data output port for the data-processing circuit).

In some embodiments, the device includes one or more mixing chambers, reaction chambers, reagents chambers, lysing chambers, washing chamber, elution chambers, extraction chambers, and/or collection chambers, where each of the chambers, if present, is in fluidic communication with the first channel. In other embodiments, the device includes at least one chamber in fluidic communication with another chamber (e.g., one or more reaction chambers in fluidic communication with at least one mixing chamber; one or more reagent chambers in fluidic communication with at least one mixing chamber and/or reaction chamber; one or more washing chambers in fluidic communication with at least one mixing chamber, reagent chamber, and/or reaction chamber).

In some embodiments, the plurality of hollow needles is configured to obtain the sample from a subject. In particular embodiments, at least one needle includes a puncturing edge (e.g., a tapered point, a sharpened bevel, or one or more prongs). In other embodiments, at least one hollow needle includes a polymer, a metal, silicon, glass, a composite material, or a combination thereof.

In some embodiments, the one or more sensing transducers is selected from an electrode (e.g., a planar electrode, a three-dimensional electrode, a porous electrode, a disk electrode, a spherical electrode, a plate electrode, a hemispherical electrode, a microelectrode, and a nanoelectrode, or an array thereof), an ion selective electrode (e.g., including a porous material and one or more ionophores), an optical sensor, an array of any of these, and combinations thereof. In particular embodiments, the sensing transducer includes an array of electrodes (e.g., optionally having a modified surface).

In some embodiments, the fluidic channel includes an array of channels configured for fluidic communication between one needle and an array of sensing transducers. In other embodiments, the array of channels is configured for fluidic communication between an array of needles and an array of sensing transducers.

In another aspect, the present invention features a device including an integral platform (e.g., a substrate), an array of one or more hollow-bored transdermal needles projecting from the platform, an array of one or more electrochemical working electrodes fixed within the platform and displaced from the needle array, and one or more fluidic channels defined within the platform, where each channel is coupled (e.g., directly or indirectly coupled) to the bore of at least one needle so as to duct sampled biological fluid therefrom, and where the one or more fluidic channels are conformed to direct sampled biological fluid from the needle array into contact with one or more electrochemical electrodes. This platform could be packaged in a modular form as well. For example, if the fluidics encompasses sample processing, the needle array and electrode array may be modular for easy disposal whereas the microfluidic sample processing is reusable. In yet another example, the needle array can be provided or configured as a disposable cartridge module (e.g., any disposable cartridge described herein). In further examples, the first channel and/or one or more transducers can be provided or configured as a detector module (e.g., any detector herein).

In one embodiment, each fluidic channel is arranged to direct the sample (e.g., sampled biological fluid) from a respective needle to one or more electrodes that are particular to the respective needle.

In some embodiments, the working electrodes include gold, indium tin oxide, and/or carbon. In yet other embodiments, the working electrodes are chemically surface-modified to facilitate the bioassay. In various embodiments, the working electrodes are chemically surface-modified to facilitate immunoassay to detect one or more protein markers (e.g., troponin and/or myoglobin).

In one embodiment, the device further includes an electrochemical reference electrode and an electrochemical counter electrode fixed within the platform.

In another embodiment, the device further includes at least one mixing chamber defined within the platform, at least one reservoir defined within the platform, and at least one controllable valve for releasing a reagent or diluent from a reservoir into a mixing chamber.

In one embodiment, the platform further includes a pump configured to facilitate the flow of the sample (e.g., sampled biological fluid) from at least one needle toward at least one working electrode.

In one embodiment, the device further includes a power source and a data-processing circuit powered by the power source and electrically connected to a counter electrode, a reference electrode, and at least one said working electrode. In some embodiments, the device further includes a data output port for the data-processing circuit.

In one embodiment, the device further includes a telemetry unit configured to receive processed data from the data-processing circuit and to transmit the data wirelessly. In various embodiments, the telemetry unit is fixed within the platform or packaged separately from the platform and connected thereto by a cable.

In another aspect, the present invention features a kit including a device of the invention (e.g., any described herein) and instructions for affixing the device to a subject and activating the device. In further embodiments, the kit includes a therapeutic agent selected from the group consisting of an anesthetic, an antiseptic, an anticoagulant, a drug, and a vaccine. In yet other embodiments, the kit includes a telemetry unit optionally including a cable.

The present invention also features methods of detecting one or more markers in a sample and/or storing one or more samples (e.g., any useful sample, such as those including blood, plasma, serum, transdermal fluid, interstitial fluid, sweat, or a bodily fluid, as well as any sample herein). In some embodiments, the method includes obtaining the sample from a subject using the device of the invention (e.g., optionally including affixing the device to the subject) and activating the device, thereby detecting one or more markers in the sample. In further embodiments, the method includes remotely relaying the results of the presence or absence of one or more markers. In yet other embodiments, the method includes storing the device, or a portion thereof (e.g., a module thereof, such as a disposable cartridge module), thereby providing a stored sample.

The present features methods of treating or diagnosing an infection in a subject (e.g., a human subject). In some embodiments, the method includes obtaining a sample from the subject (e.g., by using any device herein) and activating the device. In other embodiments, the method includes obtaining a sample from the subject, detaching the device (or a portion thereof, such as the cartridge module), reattaching the device (e.g., reattaching a detection module to the cartridge module), and then activating the device. In further embodiments, the activating step results in detecting one or more markers in the sample useful for treating or diagnosing the infection (e.g., a viral infection, such as any herein, including those that could result in a hemorrhagic fever, such as the Ebolavirus). In other embodiments, the one or more markers is any herein (e.g., an electrolyte, an ion, a signaling molecule, or any other physiologically relevant marker).

In one aspect, the present invention features a disposable cartridge (e.g., a disposable cartridge module). In some embodiments, the disposable cartridge includes a barrel including an internal volume, a distal end, and a proximal end, where the distal end of the barrel is coupled (e.g., directly coupled or indirectly coupled, such as by way of a cap structure) to a plurality of hollow needles and a substrate (e.g., any hollow needle(s) and substrate herein) and the proximal end of the barrel includes an opening; and a locking member disposed on a surface portion defining the internal volume. In other embodiments, the module further includes a sealing member disposed on a surface portion defining the internal volume.

In another aspect, the present invention features a detector (e.g., a detector module). In some embodiments, the first channel and the one or more transducers (e.g., any described herein) are configured as a detector module. In other embodiments, the detector includes a body (e.g., configured to contain the one or more transducers and the first channel), where the body includes a distal section and a proximal section; a central bore disposed within the body (e.g., and in fluidic communication with the first channel); and a mounting shaft disposed on the distal section of the body, where the mounting shaft is configured to be inserted into the opening of the disposable cartridge (e.g., any herein, including module forms thereof). In further embodiments, the detector includes a fitting structure disposed on an outer surface portion of the mounting shaft, where the fitting structure is configured to interface with the locking member of the disposable cartridge module; and/or a sealing structure disposed on an outer surface portion of the mounting shaft, where the sealing structure is configured to interface with the sealing member of the disposable cartridge module.

In yet another aspect, the present invention features a platform including a disposable cartridge module (e.g., any described herein) and a handheld module (e.g., any described herein, such as that described for a detector that is configured for handheld use). In some embodiments, the handheld module includes a body including a distal section and a proximal section; a central bore disposed within the body and in fluidic communication with the disposable cartridge module; and a mounting shaft disposed on the distal section of the body, where the mounting shaft is configured to be inserted into the opening of the disposable cartridge module. In some embodiments, the handheld module further includes a fitting structure disposed on an outer surface portion of the mounting shaft, where the fitting structure is configured to interface with the locking member of the disposable cartridge module; and/or a sealing structure disposed on an outer surface portion of the mounting shaft, where the sealing structure is configured to interface with the sealing member of the disposable cartridge module.

In other embodiments, the body further includes a handle disposed on the proximal section. In some embodiments, the body includes one or more sensing transducers (e.g., any described herein, such as one or more of an electrode and/or an ion selective electrode, including a reference electrode and/or a counter electrode) in fluidic communication with the internal volume. In other embodiments, at least one sensing transducer is configured to detect one or more markers in the sample. In other embodiments, the body further includes a pumping mechanism (e.g., a passive channel, an active pump, a vacuum source, etc.) configured to transport the sample from the hollow needles and/or the internal volume into the central bore.

In any of the embodiments herein, the platform or device further includes an electronic readout interface (e.g., a cell phone, a smartphone, etc.) configured to wirelessly communicate with the handheld module, sensing transducer(s), and/or detector (e.g., detector module). In yet other embodiment, the electronic readout interface is further configured to remotely relay the results of the presence or absence of one or more markers.

In any of the embodiments herein, at least one needle, substrate, fluidic channel, and/or sensing transducer further includes a modified surface (e.g., surface-modified with one or more capture agents, such as one or more antibodies for detecting one or more markers, enzymes, etc., as well as any described herein). In other embodiments, the modified surface includes a conductive material (e.g., a conductive polymer, such as poly(bithiophene), polyaniline, or poly(pyrrole), such as dodecylbenzenesulfonate-doped polypyrrole; a metal, such as metal nanoparticles, metal microparticles, or a metal film; or a nanotube). In yet other embodiments, the modified surface includes a linking agent (e.g., a diazonium compound, as described herein). In further embodiments, the modified surface includes a label (e.g., optionally attached to a surface by a linking agent).

In any of the embodiments herein, at least one needle (e.g., disposed within the lumen, on the interior surface, and/or on the exterior surface), substrate, fluidic channel (e.g., disposed within the channel), chamber, and/or sensing transducer (e.g., disposed on one or more electrodes, dielectrics, etc.) further includes a substance (e.g., one or more capture agents, electroactive components, linking agents, or any substance described herein).

In any of the embodiments herein, the needles, first channel, and/or transducers are, independently, provided in a high-density array. In further embodiments, the high-density array includes a modified surface (e.g., further including a linking agent, such as any described herein, including a diazonium compound).

In any of the embodiments herein, the device includes one or more components (e.g., the plurality of hollow needles, the substrate, the first channel, and the one or more transducers) integrated into a single structure (e.g., a monolithic structure, where each of the components are bonded together to form a single structure). In further embodiments, each of the components (e.g., the plurality of hollow needles, the substrate including the needles, the first channel, and the one or more transducers) is embedded in the same substrate. In further embodiments, each of the components (e.g., the plurality of hollow needles, the substrate including the needles, the first channel, and the one or more transducers) is embedded in different substrates (e.g., where the different substrates are bonded to form a multilayer device).

In any of the embodiments herein, the device includes one or more components (e.g., the plurality of hollow needles, the substrate, the first channel, and the one or more transducers) configured into separate modules (e.g., reusable or disposable modules).

In any of the embodiments herein, the device includes multiples substrates (e.g., configured in multiple layers).

In any of the embodiments herein, the device is configured in a package (e.g., a packaged chip having a housing for the device of the invention). In yet other embodiments, the device includes a sample processing module (e.g., including one or more sample chambers, valves, etc.,) in fluidic communication with a cartridge module and a detection module (e.g., a handheld module).

In any of the embodiments herein, the device further includes one or more components for relaying the presence or absence of one or more markers in the sample. Exemplary components include a data output port for the data-processing circuit, an analog-to-digital converter, a radiofrequency module, a cable, and/or a telemetry unit (e.g., configured to receive processed data from a data-processing circuit electrically connected to the transducer and to transmit the data wirelessly).

In any of the embodiments herein, the device includes one or more of a filter, a permeable or semi-permeable membrane, a valve, a chamber (e.g., any described herein, including reservoirs), a pump, a probe, a multifunctional sensor, a feedback resistor, a microscale light-emitting diode, an active/passive circuit element, an actuator, a wireless power coil, a device for radio frequency (RF) communications, a temperature sensor, a photodetector, a photovoltaic cell, a diode, and/or a liner with an adhesive layer (e.g., for affixing the device to a user).

Definitions

By “about” is meant +/−10% of any recited value.

By “fluidic communication,” as used herein, refers to any duct, channel, tube, pipe, or pathway through which a substance, such as a liquid, gas, or solid may pass substantially unrestricted when the pathway is open. When the pathway is closed, the substance is substantially restricted from passing through. Typically, limited diffusion of a substance through the material of a plate, base, and/or a substrate, which may or may not occur depending on the compositions of the substance and materials, does not constitute fluidic communication.

As used herein, “linked” or “linking” is understood to mean attached or bound by covalent bonds, non-covalent bonds, and/or linked via van der Waals forces, hydrogen bonds, and/or other intermolecular forces.

By “microfluidic” or “micro” is meant having at least one dimension that is less than 1 mm. For instance, a microfluidic structure (e.g., any structure described herein) can have a length, width, height, cross-sectional dimension, circumference, radius (e.g., external or internal radius), or diameter that is less than 1 mm. In another instance, a microneedle can have a length, width, height, cross-sectional dimension, circumference, radius (e.g., external or internal radius), or diameter that is less than 1 mm.

By “treating” a disease, disorder, or condition in a subject is meant reducing at least one symptom of the disease, disorder, or condition by administrating a therapeutic substance to the subject.

By “treating prophylactically” a disease, disorder, or condition in a subject is meant reducing the frequency of occurrence or severity of (e.g., preventing) a disease, disorder or condition by administering to the subject a therapeutic substance to the subject prior to the appearance of a disease symptom or symptoms.

By “sample” is meant any specimen obtained from a subject, a plant, an environment, a chemical material, a biological material, or a manufactured product. The sample can include any useful material, such as biological (e.g., genomic) and/or chemical matter.

By “subject” is meant a human or non-human animal (e.g., a mammal). Exemplary non-human animals include livestock (e.g., cattle, goat, sheep, pig, poultry, farm fish, etc.), domestic animals (e.g., dog, cat, etc.), or captive wild animals (e.g., a zoo animal).

### BRIEF DESCRIPTION OF THE DRAWINGS

FIG. 1 provides a perspective view of an exemplary bioassay platform according to an embodiment of the invention.

FIG. 2A-2D provides (A) a schematic view of the microneedle (MN) manifold; (B) a cross-sectional view of an exemplary MN manifold 200; (C) a perspective view of another exemplary manifold 2000 with integrated electrode arrays and fluidic channel 2005, according to an embodiment of the invention; and (D) an exploded view of another exemplary manifold 300.

FIG. 3 is a side view of an exemplary needle having measurements for the base width (labeled “1”), height from base to tip of the needle (labeled “2”), lumen width (labeled “3”), and needle thickness (i.e., distance between the exterior and interior surface, labeled “4”).

FIGS. 4 and 5 show side images of exemplary needles (scale bar is 250 μm).

FIG. 6 is a cross-sectional view of an exemplary needle having measurements for the base width (labeled “1”), base height (labeled “2”), lumen width (labeled “3”), lumen height (labeled “4”), and needle thickness (i.e., distance between the exterior and interior surface, labeled “5”).

FIG. 7 shows cross-sectional images of exemplary needles (scale bar is 250 μm).

FIG. 8 provides a non-limiting embodiment of the electrode array of FIG. 1. As seen in the figure, the electrode array includes eight working electrodes, a counter electrode, and a reference electrode (magnified view of electrodes provided in white rectangle). Exposed gold surfaces are seen, surrounded by a dielectric oxide passivation layer which defines the working area (black rectangle) that is electrochemically active. In this example, the active area has dimensions approximately 150 μm×200 μm.

FIG. 9 provides a schematic representation of an immunoassay protocol including (1) immobilization of capture or detection antibody, (2) capture of a target protein using a sandwich assay, (3) immobilization of an HRP-labeled secondary antibody, which is further labeled with linking agent “A”, and (4) catalytic turnover of a TMB substrate to produce TMB_{Ox} and an electrochemical signal. An inset of the figure illustrates an exemplary process for electrochemically immobilizing phenyl molecules by using a corresponding diazonium salt, where the phenyl molecules serve as linkers to conjugate capture antibody.

FIG. 10A is a graph showing the influence of laser energy measured at the stage relative to the fabricated voxel size for Eshell 300; a 4× microscope objective was used in this study.

FIG. 10B is a graph comparing fabrication times for a hollow microneedle (450 μm×1250 μm) with a variety of step heights between each fabricated layer. FIG. 10C is a graph showing the relationship between laser cutting speed and exit bore sizes in 2 mm thick Eshell 300 substrates.

FIG. 11 is a graph showing simultaneous electrochemical characterization of a gold electrode (n=8) array having oxide dielectric defined working electrodes with 1 mM Fe(CN)₆ in 0.1M KCl against a Ag/AgCl reference electrode and platinum wire counter electrode.

FIG. 12A is a graph characterizing electrochemical deposition of in-situ generated carboxyl diazonium on a gold electrode against an Ag/AgCl reference electrode and a platinum wire counter electrode. FIG. 12B is a graph showing optimization of carboxyl diazonium deposition parameters determined by direct secondary antibody attachment of HRP-labeled antibody, which was tested in TMB conductivity solution. Data are shown for current response (in nA) as a function of the number of cyclic voltammograms (CV) for diazonium deposition.

FIG. 13A-13B provides graphs showing calibration from an immunoassay using varying concentrations of (A) myoglobin or (B) troponin ITC complex (in ppb) and measuring the current response (in nA).

FIG. 14 is a schematic providing another exemplary bioassay platform having a microneedle manifold with integrated ion selective electrodes (ISE) and a fluidic channel, according to an embodiment of the invention. EMF is generated by the ion-to-electron process at an ISE as potassium ions from a KCl solution interact with ISE. K⁺ indicates potassium ions, Cl⁻ indicates chloride ions, and e indicates electrons.

FIG. 15A-15B shows an SEM image of (A) porous carbon and (B) porous graphene. Scale bars are 1 μm.

FIG. 16 shows reversed chronopotentiometric scans of porous graphene (PG) and porous carbon (PC) K⁺/ISE electrodes versus a planar glassy carbon substrate (GC) prepared identically. Initial scan of −1 nA for 60 s was followed by a switch to +1 nA for 60 seconds. Measurements were tested against an Ag/AgCl reference electrode and a platinum wire counter electrode in 10 mM KCl.

FIG. 17A-17C shows EMF measurements and calibration curves for porous carbon potassium ion selective electrode (PC K⁺/ISE) and porous graphene potassium ion selective electrode (PG K⁺/ISE). EMF measurement are provided for increasing KCl concentrations in solution tested against an Ag/AgCl reference electrode and a Pt wire counter electrode for (A) a PC and (B) a PG potassium ion selective electrode. The inset in (A) shows a zoomed in image of EMF response to a single KCl spike. For (A) and (B), the number on each potential step corresponds to Log of each concentration spike. FIG. 17C provides a calibration curve of a PC K⁺/ISE generated from varying concentrations of KCl tested against an Ag/AgCl reference electrode and a Pt wire counter electrode.

FIG. 18 shows EMF response for PC K⁺/ISE to alternating spikes of 5 mM KCl and 10 mM NaCl in solution tested against an Ag/AgCl reference and a Pt counter electrode. The inset shows a zoomed in view of three spikes.

FIG. 19A-19B shows (A) an image of a microfluidic chip 1900 with on-chip reference electrode 1920, ISE 1910, and counter electrode 1930 (scale bar is 10 mm) and (B) an optical image of a single hollow microneedle made with two-photon photolithography (scale bar is 250 μm).

FIG. 20A-20B shows (A) on-chip calibration of varying physiologically relevant concentrations of KCl with on-chip Ag/AgCl reference wire and Pt wire counter electrode. Solutions were drawn through the channel via a syringe and allowed to stabilize for 200 seconds. (B) Also provided is a calibration curve generated from potassium spikes on-chip.

FIG. 21A-21B shows an exemplary microfluidic chip 2100 for a sample processing module. Provided are (A) a schematic and (B) a microphotograph of the fluidic package, which contains reservoirs for cell lysing (collected from needles) and washing, as well as extraction, enzymatic cleavage and transport of DNA to an electrode array. The chip includes various chambers (labeled “1” to “6”), valves (labeled “V1” to “V3”), and a compartment for the sensor (labeled “Sensor well”).

FIG. 22 shows a schematic of an exemplary handheld diagnostic platform 2200, which includes a detector module with electronic readout (upper left) for use with a disposable microneedle cartridge 2270. Also shown are a cross-sectional view of this platform (right) and a plan view of a tray including such disposable cartridges (bottom).

FIG. 23 shows a schematic of an exemplary handheld sample acquisition platform 2300, which includes an acquisition module (upper left) for use with a disposable microneedle cartridge 2370. Also shown are a cross-sectional view of this platform (right) and a plan view of a tray including such disposable cartridges (bottom).

FIG. 24 shows schematics of an exemplary disposable cartridge 2400 for use with a mounting shaft 2460 of a handheld module.

FIG. 25A-25B provides schematics of another exemplary disposable cartridge 2500 for use with a mounting shaft 2560 of a handheld module.

### DETAILED DESCRIPTION OF THE INVENTION

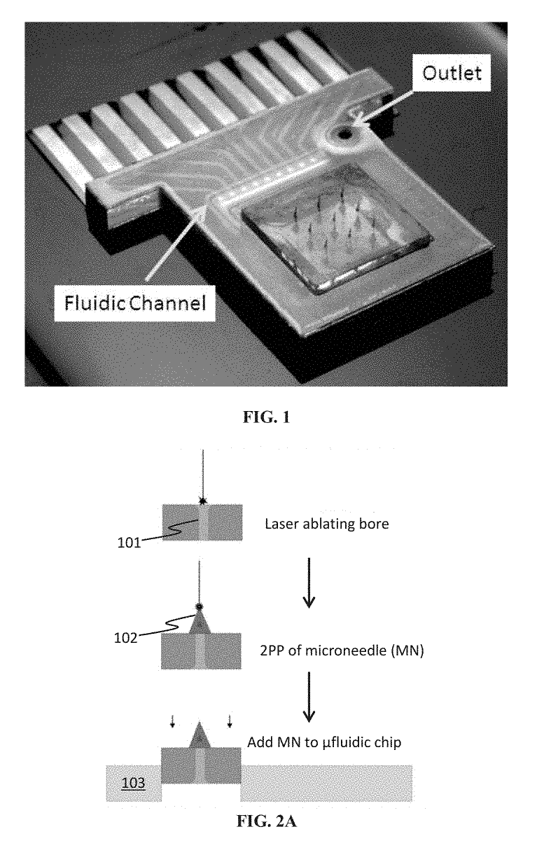

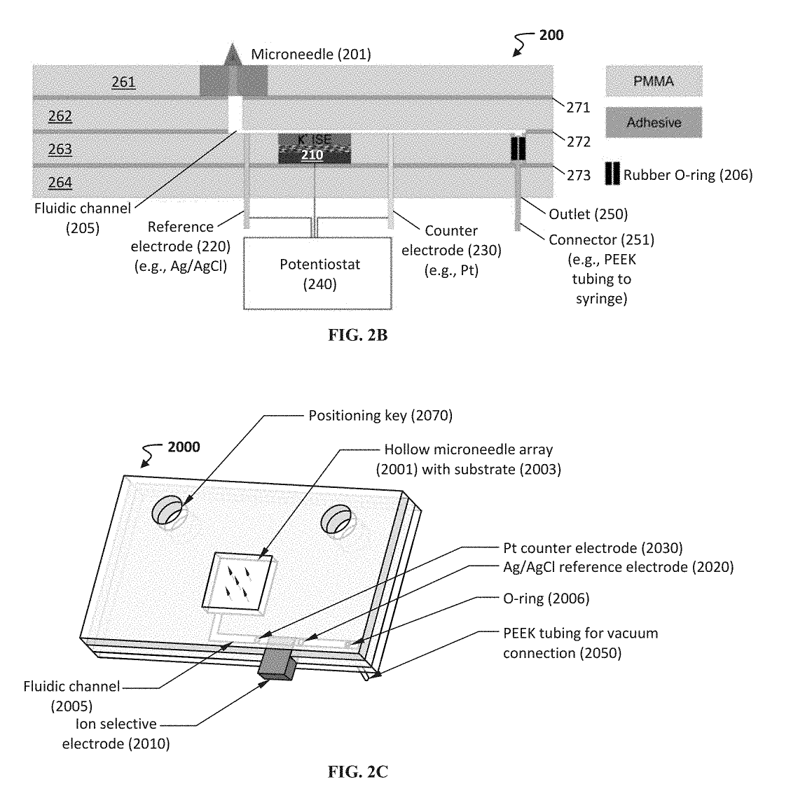

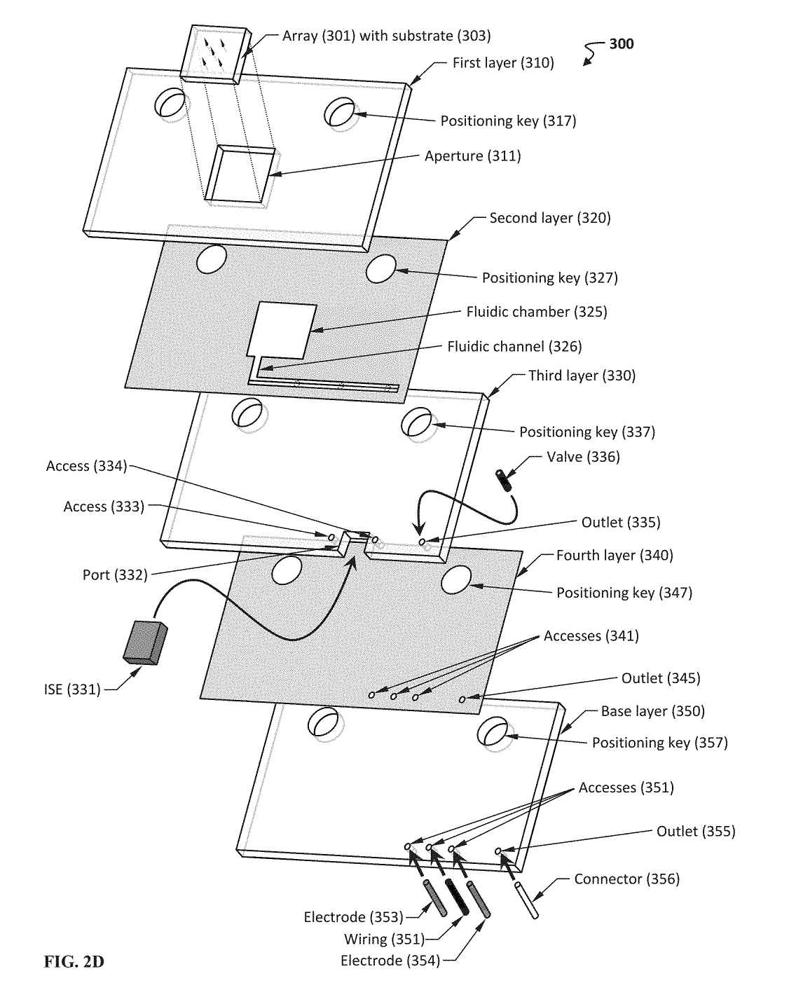

We developed a hollow microneedle manifold integrated on the same platform with an electrode array and a fluidic channel. In one example, we fabricated an eight-channel electrode array using photolithographic patterning and dielectric insulating layers to expose a 112 μm wide by 150 μm gold working area. The resulting chip was packaged using plastic laminate technology with a fluidic channel that could access the microneedles and flow solution over the electrode array. This type of device is a significant advancement towards an autonomous microneedle platform, which is capable of transdermally accessing interstitial fluid and performing real time and repeated measurements for a variety of physiologically relevant analytes. A non-limiting example of our device is illustrated in FIG. 1 and FIG. 2A-2D.

More specifically, a microfluidic manifold was constructed from acrylic sheets and medical grade pressure sensitive adhesive (PSA, e.g., Mylar® adhesives, which is a polyethylene terephthalate film) using a precision cutting laser. The acrylic sheets were typically 2 mm in thickness, although thinner or thicker sheets could be used depending on the particular application contemplated, the mechanical robustness associated with the intended function, and the form factor. Medical grade PSA was chosen because it is frequently used in the construction of commercial bioassay devices; it has demonstrated low outgassing, low chemical leaching, and biocompatibility.

Each of the materials used for construction was cut with a laser and sequentially assembled on a jig to create complex fluidic networks, with lateral flow channels being formed in the adhesive layers and connecting vias being formed in the acrylic sheet. After the layers were stacked and assembled, they were pressed together for 2 minutes at 500 psi to assure adhesion of the laminate layers. In one example, the design has one acrylic layer and two adhesive layers. The bottom adhesive layer forms the flow channel on the surface of the electrochemistry sensing chip, and the top adhesive layer seals the microneedles to the laminate cartridge. Based on the desired functionality, a skilled artisan would be able to include additional layers and structures to control flow of the sample, reagents, etc.

For instance, multilayered devices are provided in FIG. 2B-2D. As can be seen, in one instance, the device 200 can include a plurality of polymer substrates 261-264 and a plurality of adhesive layers 271-273, where each adhesive layer is disposed between two polymer substrates. Fluidic connections, by way of channels, inlets, outlets, or vias, can be formed either within a polymer substrate and/or an adhesive layer. As can be seen in FIG. 2B, the fluidic channel 205 is formed by an inlet disposed in polymer substrate 262 and a gap in the adhesive layer 272.

The device 200 includes many components, including a microneedle 201 disposed in a substrate, a fluidic channel 205 coupled to the substrate, an ion selective electrode 210, a sealing member 206 disposed in a portion of the outlet 250, and a connector 251 in fluidic communication with the outlet 250. The device can include any other useful sensing transducers, such as a reference electrode 220 and a counter electrode 230 optionally connected to a potentiostat 240.

The device can be assembled in any useful manner. For instance, FIG. 2C shows a perspective view of an exemplary device 2000 employing a positioning key 2070 to align various layers during fabrication. Also provided are the microneedle array 2001 disposed in a substrate 2003, a fluidic channel 2005, and the following transducers in fluidic communication with this fluidic channel: a positionable (and optionally replaceable) sensing transducer (e.g., an ion selective electrode 2010), a counter electrode (e.g., a Pt counter electrode 2030), and a reference electrode (e.g., a Ag/AgCl reference electrode 2020).

The device also includes an outlet placed at the end of the fluidic channel, where the outlet includes an O-ring sealing member 2006 and a tubing connector 2050 (e.g., for optional connection to a pumping mechanism, such as a vacuum source). In one instance, for external fluid connections, we enclosed conventional Viton rubber O-rings (size 001) within the cartridge. Inserting 1/32″ tubing into these captured O-rings can create a fluid-tight seal so that the application of positive or negative pressure can cause fluid to be injected or drawn through the microneedle array. A cross-sectional view of one example of such a microfluidic manifold is provided by FIG. 2C.

FIG. 2D provides an exploded view of an exemplary device 300. The device includes a plurality of layers 310-350 (e.g., layers formed from a polymer and/or an adhesive, such as any herein). The first layer 310 includes a positioning key 317, as well as an aperture 311 configured to include a microneedle array 301 disposed on a substrate 303. The second layer 320 is optionally an adhesive layer and includes a positioning key 327 (aligned to positioning key 317), a fluidic chamber 325 configured to be in fluidic communication with the hollow lumen of the microneedle array 301, and a fluidic channel 326 in fluidic communication with the fluidic chamber 325.

The third layer 330 includes one or more features to align and place one or more sensing transducers. As can be seen, this layer 330 includes a port 332 and a plurality of accesses 333, 334, which are configured to place a sensing transducer (e.g., an ion selective electrode 331) in the fluidic path provided by the fluidic channel 326 (the dashed lines in channel 326 notes the positions of the transducers when the layers are aligned). This layer also includes an outlet 335 configured to interface with a valve 336 and to be aligned with the fluidic channel 326, as well as a positioning key 337. The fourth layer 340 includes a positioning key 347, an outlet 345 in fluidic communication with the outlet 335 in the third layer, and a plurality of accesses 341 in fluidic communication with the port 332 and accesses 333, 334 in the third layer.

The base layer 350 includes a positioning key 357, an outlet 355 in fluidic communication with the outlet 345 in the fourth layer, and a plurality of accesses 351 in fluidic communication with the accesses 341 in the third layer. The outlets and accesses in this base layer facilitates insertion of one or more wiring 351, electrodes 353, 354, and/or fluidic connectors 356. A skilled artisan would understand that additional modifications and design consideration can be implemented to achieve the desired fluidic network or path.

The manifold can include hollow microneedles prepared in any useful manner. For instance, as shown in FIG. 2A, a laser is used to ablate an inlet 101 into a substrate. Then, a microneedle 102 is formed, where the hollow lumen of the microneedle is in fluidic communication with the inlet 101. In one instance, this step is performed by using two-photon polymerization, as described herein. Then, finally, the microfluidic manifold including the hollow microneedle and substrate is coupled to a first channel disposed in a microfluidic chip 103.

In one instance, hollow microneedles were prepared using a laser direct write system utilizing two-photon polymerization (2PP). First, a CAD file was created in the desired shape and dimensions of the microneedle and was uploaded to the LDW operating software (GOLD3D). The software sliced the CAD file and assigned laser and writing parameters such that the fabrication process can be optimized.

The two-photon polymerization effect is achieved with the help of Ti:Sapphire laser, which was operated at 800 nm, 150 fs, and 76 MHz. Eshell 300 was used as the resin for both the hollow microneedles and the substrates. The substrate can be formed by any useful process, e.g., such as any described herein. The process can include, without limitation, stereolithography, 2PP, etc., including any combinations thereof.

A first substrate can be used as a “base” to fabricate the needle, lancet, or puncturing mechanism onto and to be later integrated into a second substrate for the microfluidic chip. The first substrate is made so that it either fits within a recess on the microfluidic chip or acts as the top layer of the microfluidic chip. Furthermore, the transducer can be formed in a third substrate, which is integrated with the chip having one or more fluidic channels. Alternatively, the needles, microfluidic chip, and transducers are formed in the same substrate.

Substrates are made either with the 2PP system, a stereolithography system, by molding, by casting, or any other useful method. In particular, the 2PP system allows us to selectively polymerize a resin based on a CAD file to create the microneedles, and we choose a substrate made from the same material or similar material so that the chemical bonds between the microneedle and substrate are the same, which creates a strong bond between the two.

The substrates were created in PDMS molds made by laser cutting PMMA to 10 mm×10 mm×2 mm pieces and molding them with PDMS. Eshell 300 was then placed in the molds and cured with a UV lamp. In order to make a fluidic connection between the microfluidic chip and the hollow microneedle, a bore was cut into the substrates with a CO₂ laser such that the bore diameter was around 150 μm. A well was made on top of the bore-containing substrate such that a microneedle could be written onto the substrate.

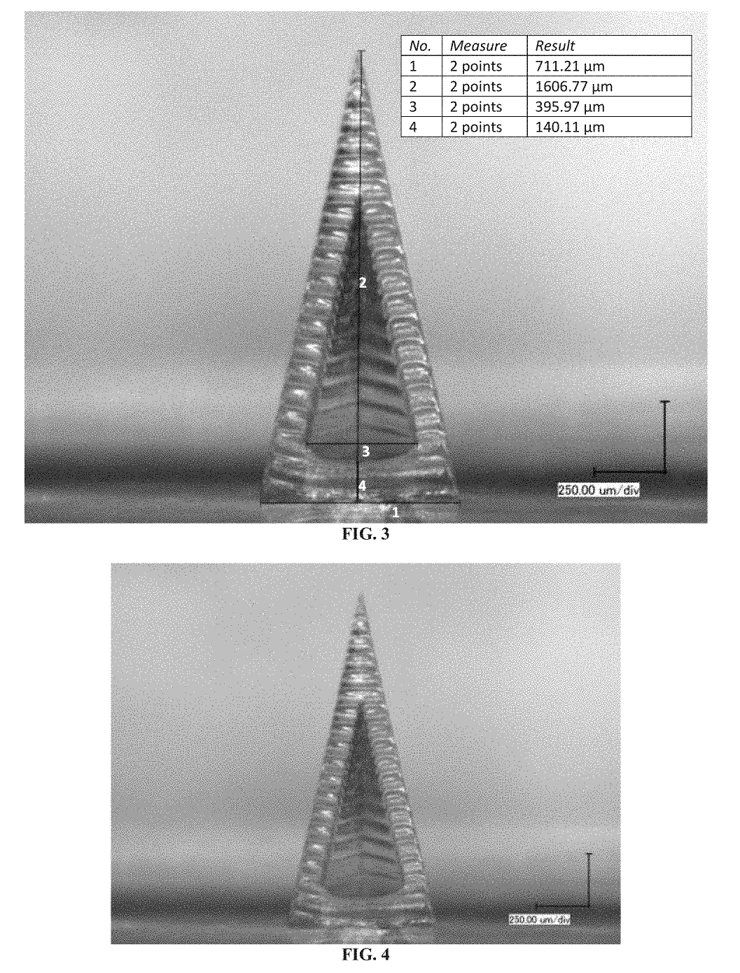

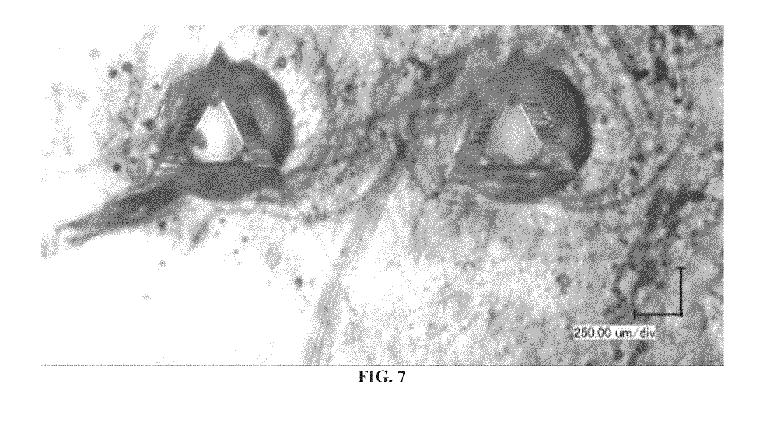

FIGS. 3-7 provide various views of examples of fabricated microneedles.

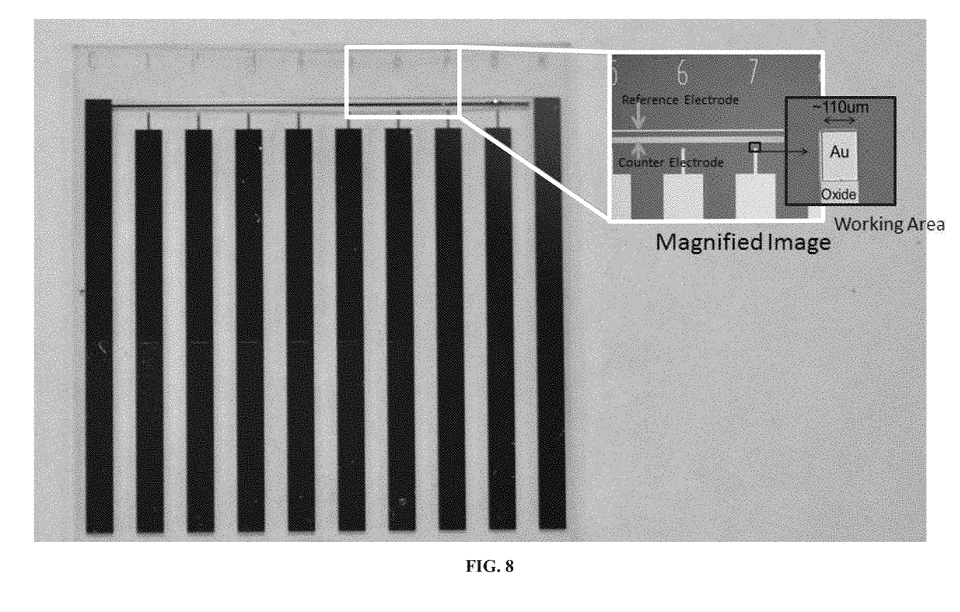

FIG. 8 provides, among other things, a view of an exemplary electrode array including eight working electrodes, a counter electrode, and a reference electrode. The eight working elements were integrated with the microneedle array.

Six-inch-diameter glass wafers were used as substrates for the electrode arrays. Standard photolithography techniques were used to pattern 150 Å Cr/3000 521 Au electrodes and contact pads. In order to precisely define the electrode surface area, a 2000 Å thick silicon nitride layer was deposited at 350° C. over the entire device using PECVD. A photolithography step defined a precise opening over the dielectric layer, which measured 112 μm wide by 150 μm high. An SF₆ plasma etch was then used to selectively remove the exposed silicon nitride until the Au layer underneath was reached.

FIG. 8 shows the 1120 μm×150 μm Au working area, which is the only part of the electrode that was exposed to solution and was electrochemically active. Also shown are the counter and reference electrodes, which were patterned on the chip. The devices were then cut using a dicing saw.

The electrode array of FIG. 8 was optimized for immunoassays to detect either troponin or myoglobin. Troponin and myoglobin are used in the clinical setting as biomarkers for detection of cardiac and skeletal muscle injuries, respectively. This approach included a sandwich antibody assay, consisting of a capture antibody and a secondary detection antibody labeled with a horseradish peroxidase enzyme that catalyzes conversion of a 3,3′,5,5′-tetramethylbenzidine (TMB) substrate to an electrochemically-detectable product.

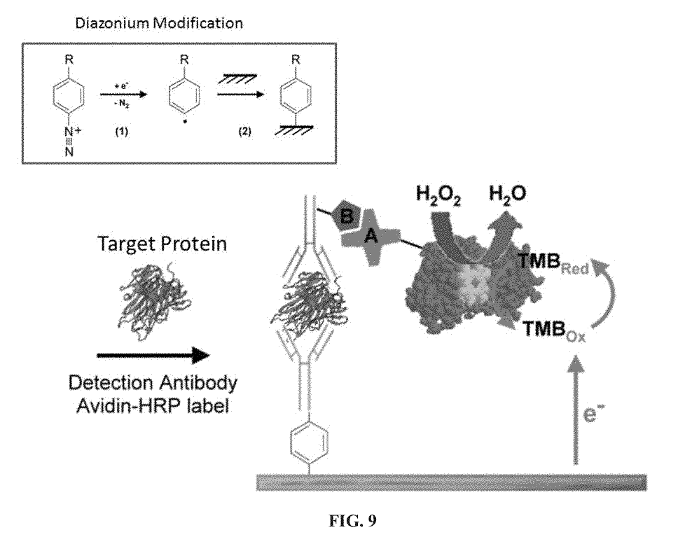

FIG. 9 provides a schematic representation of an immunoassay protocol including a sandwich assay, as well as surface-modified electrodes. In particular, the electrodes were initially modified with phenyl molecules to immobilize capture antibodies by electrochemical reduction from the corresponding phenyl diazonium molecules.

Needles

The device of the invention can have one or more needles of any useful dimension, such as length, width, height, circumference, and/or cross-sectional dimension. In particular, a skilled artisan would be able to optimize the needle length based on the type of fluid or type of tissue to be measured. For instance, the skin can be approximated as two layers including the epidermis (thickness of 0.05 to 1.5 mm) and the dermis (thickness of 0.3 to 3 mm). Accordingly, to obtain fluid in the dermis layer, the needle can be optimized to have a length that is more than about 0.3 mm, 0.5 mm, 1 mm, 1.5 mm, 2 mm, 2.5 mm, or 3 mm, depending on the desired location of the device on the body. A desired cross-sectional dimension can be determined by the skin site to be sampled (e.g., a dimension to allow for local testing of the subject, while minimizing pain), by the desired flow rate of the sample within the lumen of the needle (e.g., the flow rate can be optimized to allow for obtaining a fluid within a particular sampling time, or to minimize sample contamination, coagulation, and/or discomfort to the subject), by the desired volume of sample to be collected, etc.

To access a sample within a subject, each needle can have one or more puncturing edges of any useful geometry. In some embodiments, the puncturing edge at the distal end of the needle includes a tapered point. In particular embodiments, the tapered point is located at the apex of a pyramidal needle, where the base of the needle is attached to the substrate and one side of the pyramidal needle is open, thereby forming the lumen of the needle. Exemplary pyramidal needles are provided in FIGS. 3-7 herein. In yet other embodiments, the puncturing edge is a sharpened bevel for any useful geometrical shape forming the hollow needle, such as a cylinder, a cone, a post, a rectangle, a square, a trapezoid, as well as tapered forms thereof (e.g., a tapered cylinder or a tapered post), etc. In further embodiments, the puncturing edge includes one or more prongs (e.g., two, three, four, five, or more prongs) for obtaining a sample from a subject.

The needles can be formed from any useful material, e.g., a polymer (e.g., such as a biocompatible polymer; an acrylate-based polymer, such as e-Shell 200 (0.5-1.5% wt phenylbis(2,4,6 trimethylbenzoyl)-phosphine oxide photoinitiator, 15-30% wt propylated (2) neopentyl glycoldiacrylate, and 60-80% wt urethane dimethacrylate) or e-Shell 300 (10-25% wt urethane dimethacrylate and 10-20% tetrahydrofurfuryl-2-methacrylate); a resorbable polymer, e.g., polyglycolic acid (PGA), polylactic acid (PLA) including poly(L-lactide) (PLLA) and poly(D-lactide) (PDLA), or PGA-PLA copolymers; or any described herein), silicon, glass, a metal (e.g., stainless steel, titanium, aluminum, or nickel, as well as alloys thereof), a composite material, etc. The surface (e.g., interior and/or exterior surface) of the needle can be surface-modified with any agent described herein (e.g., a linking agent, capture agent, label, and/or porous material, as described herein). Additional surface-modified needles are described in U.S. Pub. No. 2011/0224515, as well as U.S. Pat. Nos. 7,344,499 and 6,908,453, each of which is incorporated by reference herein in its entirety.

The needles can be formed from any useful process. For instance, when formed from a polymer, the needle can be formed by two-photon polymerization (2PP), as described, e.g., in Gittard S D et al., “Fabrication of polymer microneedles using a two-photon polymerization and micromolding process,” J. Diabetes Sci. Technol. 2009; 3:304-11, which is incorporated by reference in its entirety. Additional methods include polymerizing, molding (e.g., melt-molding), spinning, depositing, casting (e.g., melt-casting), etc. Methods of making needles are described in U.S. Pat. Nos. 7,344,499 and 6,908,453, each of which is incorporated by reference herein in its entirety.

Furthermore, a plurality of needles can be provided in an array. The array can include two, three, four, five, six, seven, eight, nine, ten, fifteen, twenty, or more needles configured in any useful arrangement (e.g., geometrical arrangements). The array can have any useful spatial distribution of needles (e.g., a square, rectangular, circular, or triangular array), a random distribution, or the like.

The needle can include any useful substance, e.g., any described herein. In particular embodiments, one or more needles includes a substance that further includes one or more capture agents. For example, the needle can include (e.g., within a portion of the lumen of the needle) a matrix including an electroactive component. The electroactive component can be, e.g., a carbon paste including one or more capture agents (e.g., an enzyme or a catalyst (e.g., rhodium) for detecting a marker). Further embodiments are described in Windmiller J R et al., “Microneedle array-based carbon paste amperometric sensors and biosensors,” Analyst 2011; 136:1846-51, which is incorporated by reference in its entirety.

Exemplary needles are described in U.S. Pub. No. 2011/0224515; and Int. Pub. No. WO 2013/058879, each of which is incorporated by reference in its entirety.

Transducers

The transducer can be any useful structure for detecting, sensing, and/or measuring a marker or target of interest. Exemplary transducers include one or more of the following: optical sensors (e.g., including measuring one or more of fluorescence spectroscopy, interferometry, reflectance, chemiluminescence, light scattering, surface plasmon resonance, or refractive index), piezoelectric sensors (e.g., including one or more quartz crystals or quartz crystal microbalance), electrochemical sensors (e.g., one or more of carbon nanotubes, electrodes, field-effect transistors, etc.), etc., as well as any selected from the group consisting of an ion selective electrode, an ion sensitive field effect transistor (e.g., a n-p-n type sensor), a light addressable potentiometric sensor, an amperometric sensor (e.g., having a two-electrode configuration (including reference and working electrodes) or a three-electrode configuration (including reference, working, and auxiliary electrodes)), and/or an impedimetric sensor.

In particular embodiments, the transducer is a working electrode having an exposed working area. The working electrode includes any useful conductive material (e.g., gold, indium tin oxide, titanium, and/or carbon). Optionally, the working area is surface modified, e.g., with a linking agent and/or a capture agent described herein. These transducers can include one or more other components that allows for detection, such as a ground electrode, a reference electrode, a counter electrode, a potentiostat, etc. The electrode can have any useful configuration, such as, e.g., a disk electrode, a spherical electrode, a plate electrode, a hemispherical electrode, a microelectrode, or a nanoelectrode; and can be formed from any useful material, such as gold, indium tin oxide, carbon, titanium, platinum, etc.

Exemplary electrodes include a planar electrode, a three-dimensional electrode, a porous electrode, a post electrode, a microelectrode (e.g., having a critical dimension on the range of 1 to 1000 μm, such as a radium, width, or length from about 1 to 1000 μm), a nanoelectrode (e.g., having a critical dimension on the range of 1 to 100 nm, such as a radium, width, or length from about 1 to 100 nm), as well as arrays thereof. For instance, a three-dimensional (3D) electrode can be a three-dimensional structure having dimensions defined by interferometric lithography and/or photolithography. Such 3D electrodes can include a porous carbon substrate. Exemplary 3D porous electrodes and methods for making such electrodes are described in U.S. Pat. No. 8,349,547, which is incorporated herein by reference in its entirety. In another embodiment, the electrode is a porous electrode having a controlled pore size (e.g., a pore size less than about 1 μm or about 0.1 μm). In some embodiments, the electrode is a post electrode that is a carbon electrode (e.g., formed from a photoresist (e.g., an epoxy-based resist, such as SU-8) that has been pyrolyzed), which can be optionally modified by depositing a conductive material (e.g., a conductive polymer or a metal, such as any described herein). In yet other embodiments, the electrode is a nanoelectrode including a nanodisc, a nanoneedle, a nanoband, a nanoelectrode ensemble, a nanoelectrode array, a nanotube (e.g., a carbon nanotube), a nanopore, as well as arrays thereof. Exemplary nanoelectrodes are described in Arrigan D W M, Analyst 2004; 129:1157-65, which is incorporated by reference herein in its entirety.

Any of these electrodes can be further functionalized with a conductive material, such as a conductive polymer, such as any described herein, including poly(bithiophene), polyaniline, or poly(pyrrole), such as dodecylbenzenesulfonate-doped polypyrrole; a metal, such as metal nanoparticles (e.g., gold, silver, platinum, and/or palladium nanoparticles), metal microparticles, a metal film (e.g., palladium or platinum), etc.; a nanotube; etc. Additional electrodes are described in Int. Pub. No. WO 2013/058879 and U.S. Pat. No. 8,349,547, each of which is incorporated herein by reference in its entirety.

The needles and transducers can be configured in any useful manner. For instance, the needles and transducers can be fluidically connected by a fluidic channel. In other embodiments, the needle can include a transducer within the lumen of a needle, such as those described in Int. Pub. No. WO 2013/058879, which is incorporated by reference in its entirety. In some embodiments, the needle can include a transducer on the exterior surface of the needle. For instance, the transducer can include one or more conductive layers on the exterior surface of the needle, where the conductive layer can include one or more capture agents (e.g., any described herein). Such needles and conductive layers, as well as sensing layers and protective layers, are described in, e.g., Int. Pub. No. WO 2006/116242, which is incorporated herein by reference in its entirety.

The transducer can be integrated with the needle by any useful process and with any useful configuration. For example, the transducer can be a carbon fiber electrode configured to reside within the lumen of a needle. Such a configuration is described, e.g., in Miller P R et al., “Integrated carbon fiber electrodes within hollow polymer microneedles for transdermal electrochemical sensing,” Biomicrofluidics 2011; 5:013415 (14 pages), which is incorporated herein by reference in its entirety.

The present invention could also allow for integration between one or more needles with an array of transducers. The needle and electrode can be configured in any useful way. For instance, each needle can be associated with a particular electrode, such that there is a one-to-one correspondence between the fluid withdrawn into the needle and the fluid being delivered to the electrode. In other embodiments, each needle is associated with an array of electrodes. In yet other embodiments, an array of needles is associated with an individual electrode or with an array of electrodes.

The fluidic connection between the needle and the electrode can be established by a channel or a network of channels. In one non-limiting example, when one needle is associated with an array N×M of electrodes, a network containing channels can be interfaced between the needle and electrode array. Such a network can include a main channel that splits into N sub-channels, which in turn split into M smaller channels. A skilled artisan would understand how to optimize channel geometry to fluidically connect one or more needles to one or more electrodes.

In some embodiments, the array is a high density array including N×M array of electrodes, where each electrode can be individually addressable. In further embodiments, the high density array is surface modified with one or more capture agents and/or one or more linking agents, as described herein. Exemplary values for N and M include, independently, 1, 2, 3, 4,5, 6, 7, 8, 9, 10, 12, 15, 20, 25, 30, 40, 50, 75, 100, etc.

The transducers can optionally be surface-modified with one or more capture agents (e.g., one or more antibodies for detecting one or more markers, such as any described herein). Such transducer can include, e.g., an ion selective electrode (ISE) for detecting one or more ions. An ISE can include a porous material and one or more capture agents, such as, e.g., one or more ionophores. Exemplary porous materials include porous carbon, graphene, silicon, conducting polymer (e.g., such as any described herein), etc. Exemplary ionophores include one or more of the following: a crown ether, a macrocyclic compound, a cryptand, a calixarene, A23187 (for Ca²⁺), beauvericin (for Ca²⁻, Ba²⁺), calcimycine (for A23187), enniatin (for ammonium), gramicidin A (for H⁺, Na⁻, K⁺), ionomycin (for Ca²⁺), lasalocid, monensin (for Na⁺, H−), nigericin (for K⁺, H⁺, Pb²⁺), nonactin (for ammonium), nystatin, salinomycin (for K⁺), valinomycin (for K¹), siderophore (for Fe³⁺), etc. Such materials and ISEs can be obtained by any useful process, such as templating (see, e.g., Lai C et al., Anal. Chem. 2007; 79:4621-6, which is incorporated herein by reference in its entirety), interference lithography, molding, casting, spinning, electrospinning, and/or depositing.

Another exemplary transducer includes a detection electrode configured for a sandwich assay. Such an electrode include, e.g., a conductive surface and a first capture agent (e.g., an antibody) immobilized on the conductive surface, where the first capture agent is optionally attached by a linking agent. In use, the marker of interest binds to the first capture agent to form a complex, and further capture agents can be used to bind the resultant complex. To detect the complex, further capture agents can include a detectable label or an enzyme that reacts with an agent to provide a detectable signal (e.g., an agent that is a fluorogenic, enzyme-cleavable molecule).

Substrate

In general, a substrate refers to a substantially planar surface or media containing one or more structures. For instance, one or more needles, fluidic channels, and/or transducers can be embedded in the same substrate or in different substrates. The substrate can be formed from any useful material. Exemplary materials include any described herein, such as a flexible substrate (e.g., a polyvinylacetate, a polyester, or any other described herein).

The substrate can include one or more inlets in fluidic communication with the needle. In this manner, a sample collected within the needle can be delivered through the needle and into the inlet. Generally, the inlet is further configured to be in fluidic communication with one or more fluidic channels, as described herein. Such fluidic channels allow the sample to be delivered to one or more sensing transducers, thereby detecting the marker of interest.

Other structures can be integrated into a substrate, such as, e.g., a filter, a permeable or semi-permeable membrane, a valve, and/or an electrode (e.g., any described herein).

Furthermore, the device of the invention can include multiple substrates (e.g., configured in multiple layers). For ease of manufacturing, the needles can be manufactured in a first substrate, other structures (e.g., fluidic channels) can be included in a second substrate, and the transducer(s) can be included in a third substrate. Then, the first, second, and third substrates are aligned (e.g., by including one or more registration marks or alignment holes on each substrate) and then laminated (e.g., by using an adhesive layer between substrate layers). A skilled artisan would be able to optimize manufacturing parameters for the particular design of the device and arrangement of these various structures.

Fluidic Channels

One or more fluidic channels (including inlets) can be used to effect fluidic communication between two structures or regions.

Any of the fluidic channels described herein can be surface modified (e.g., to increase biocompatibility, decrease protein adsorption or absorption, and/or decrease surface contamination). Furthermore, such fluidic channels can also include one or more capture agents to selectively or non-selectively bind to cellular components or contaminants within a sample.

Surface Modification

Any of the surfaces described herein may be modified to promote biocompatibility, to functionalize a surface (e.g., using one or more capture agents including the optional use of any linking agent), or both. Exemplary surfaces include those for one or more transducers, needles, fluidic channels, filters, and/or substrates.

The surface can be modified with any useful agent, such as any described herein. Exemplary agents include a capture agent (e.g., any described herein, such as an antibody); a polymer, such as a conducting polymer (e.g., poly(pyrrole), poly(aniline), poly(3-octylthiophene), or poly(thiophene)), an antifouling polymer, or a biocompatible polymer (e.g., chitosan), or a cationic polymer)); a coating, e.g., a copolymer, such as a copolymer of an acrylate and a lipid, such as butyl methacrylate and 2-methacryloyloxyethyl phosphorylcholine; a film; a label (e.g., any described herein); a linking agent (e.g., any described herein); an electroactive component, such as one or more carbon nanotubes or nanoparticles (e.g., gold, copper, cupric oxide, silver, or platinum nanoparticles), such as, for stabilizing an electrode; an enzyme, such as glucose oxidase, cholesterol oxidase, horse radish peroxidase, or any enzyme useful for oxidizing, reducing, and/or reacting with a marker of interest; or combinations thereof (e.g., an electroactive component coated with a polymer, such as a carbon nanotube coated with polyaniline).

Optionally, linking agents can be used be attach the agent to the surface. Exemplary linking agents include compounds including one or more first functional groups, a linker, and one or more second functional groups. In some embodiments, the first functional group allows for linking between a surface and the linker, and the second functional group allows for linking between the linker and the agent (e.g., a capture agent, a label, or any agent described herein). Exemplary linkers include any useful linker, such as polyethylene glycol, an alkane, and/or a carbocyclic ring (e.g., an aromatic ring, such as a phenyl group). In particular embodiments, the linking agent is a diazonium compound, where the first functional group is a diazo group (−N₂), the linker is an aryl group (e.g., a mono-, bicyclic, or multicyclic carbocyclic ring system having one or two aromatic rings and is exemplified by phenyl, naphthyl, xylyl, 1,2-dihydronaphthyl, 1,2,3,4-tetrahydronaphthyl, fluorenyl, indanyl, indenyl, and the like), and the second functional group is a reactive group for attaching a capture agent or a label (e.g., where the second functional group is halo, carboxyl, amino, sulfo, etc.). Such diazonium compounds can be used to graft an agent onto a surface (e.g., an electrode having a silicon, iron, cobalt, nickel, platinum, palladium, zinc, copper, or gold surface). In some embodiments, the linking agent is a 4-carboxybenzenediazonium salt, which is reacted with a capture agent by 1-ethyl-3-[3-dimethylaminopropyl]carbodiimide hydrochloride (EDC)/N-hydroxysuccinimide (NHS) crosslinking, to produce a diazonium-capture agent complex. Then, this resultant complex is deposited or grafted onto a surface (e.g., an electrode surface).

Other exemplary linking agents include pairs of linking agents that allow for binding between two different components. For instance, biotin and streptavidin react with each other to form a non-covalent bond, and this pair can be used to bind particular components. As shown in FIG. 9, e.g., a first capture agent is an antibody attached to a substrate with a diazonium linking agent, a second capture agent is an antibody labeled with biotin (labeled “B”), and a third capture agent is an enzyme labeled with streptavidin (labeled “A”).

Platform

The present invention also includes a platform, which in turn includes a cartridge module (e.g., a disposable cartridge module) and a handheld module (e.g., a handheld detector module, diagnostic module, or acquisition module). In particular, the cartridge and handheld modules are designed to have matching configurations, thereby allowing the cartridge to be replaced with minimal effort by the user. The handheld module can be adapted for any useful purpose. For instance, when the platform is to be used for diagnosing or treating a disease or a medical condition, then the handheld module can include one or more detectors or electronic devices for real-time detection of one or more markers. Alternatively, when the platform is to be used for acquiring samples, then the handheld module can include one or more pumping mechanisms (e.g., active or passive pumps or pressure sources) to draw an obtained sample through the hollow microneedles and into the cartridge.

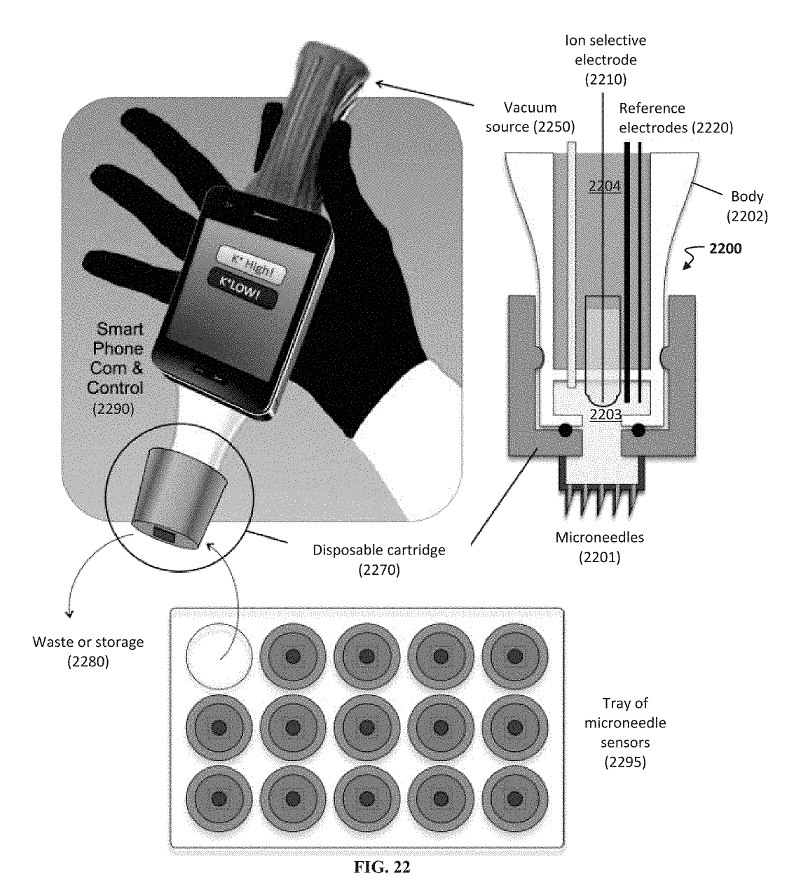

FIG. 22 provides an exemplary diagnostic platform 2200. The platform includes a handheld module, which includes a body 2202, and a cartridge module (e.g., a disposable cartridge 2270). The handheld module can include one or more transducers and/or pumping mechanisms disposed within the body. In addition, the handheld module can be integrated with an electronic readout or, alternatively, can be configured to wirelessly communicate with an external device that provides such a readout (such as an electronic readout interface, including a smartphone, a cell phone, a mobile device, a mobile phone, etc.).

As seen in FIG. 22, the handheld module includes a body 2202 having a central bore 2203 in fluidic communication with the disposable cartridge 2270. The body also includes an isolated region 2204 within the bore 2203 that contains the sensing transducers 2210, 2220 (e.g., an ISE, a reference electrode, or any described herein) and the pumping mechanism 2250 (e.g., a vacuum source). The distal portion of the body 2202 includes a mounting shaft configured to interface with the disposable cartridge 2270. The proximal portion of the body 2202 includes a handle, and the body can be configured to interface with an electronic readout interface 2290, such as a smartphone, to control, e.g., the detector, the pumping mechanism, and/or release mechanism that detaches the cartridge from the handheld module.

The cartridge 2270 includes a plurality of microneedles 2201 disposed on the distal end. In some embodiments, a plurality of cartridges is provided as a tray 2295 of sensors. After use with the handheld module, the cartridge containing the sample can either be discarded or stored for later analysis 2280.

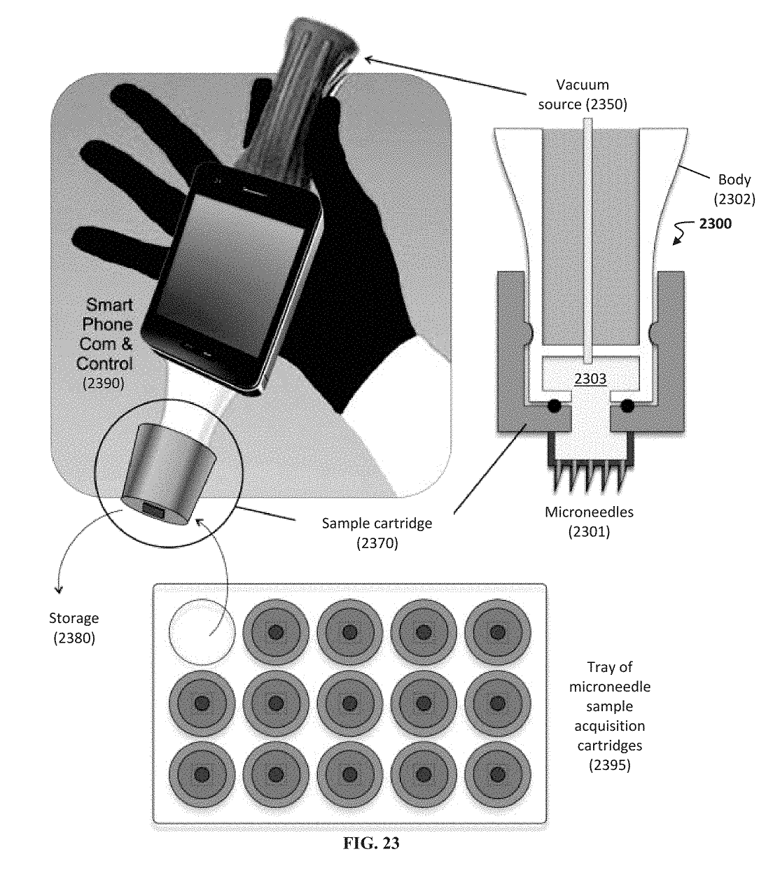

FIG. 23 provides an alternative embodiment for a sample acquisition platform 2300. Here, the handheld module includes a body 2302 and a pumping mechanism 2350 (e.g., a vacuum source) in the central bore 2303, which in turn is configured to be in fluidic communication with the sample cartridge 2370 having a plurality of microneedles 2301. Again, the handheld module can be configured to communicate with an electronic device 2390 to control, e.g., the pumping mechanism and/or release mechanism that detaches the cartridge from the handheld module. After use with the handheld module, the cartridge containing the sample is stored for later analysis 2380. In some embodiments, a plurality of cartridges is provided as a tray 2395 of acquisition cartridges.

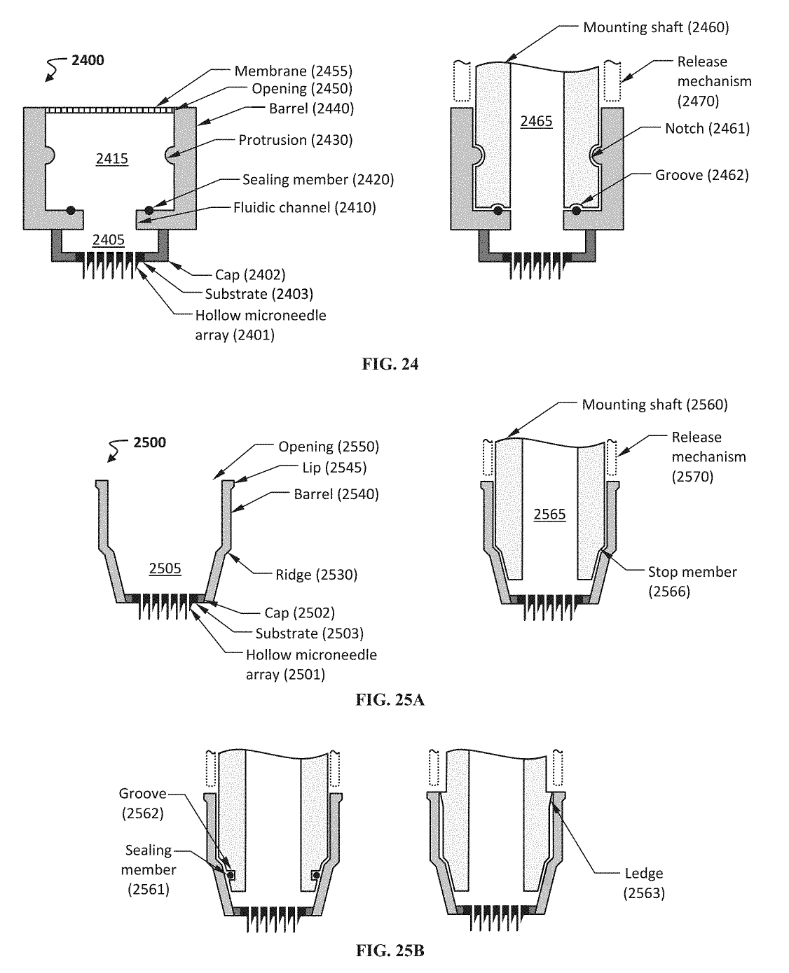

The platform can include any useful structure(s) to affix the cartridge to the handheld module and then, after use, to release the cartridge for disposal for storage. For instance, the cartridge and handheld module can include one or more locking members and structures to position the cartridge at the distal end of the handheld module; sealing members and structures to ensure a fluidic seal between the modules, thereby containing the sample in a controlled manner; and/or release mechanisms to release the cartridge from the distal end of the handheld module. Exemplary cartridges and matching configurations for the handheld module are provided in FIG. 24 and FIG. 25A-25B.

FIG. 24 provides an exemplary cartridge 2400 including a barrel 2440 and a cap 2402 configured to house the substrate 2403 and microneedle array 2401. The cap 2402 is disposed at the distal end of the barrel 2440, and an opening 2450 is disposed at the proximal end of the barrel 2440. In particular, the opening 2450 is configured to interface with the handheld module (e.g., the mounting shaft of the handheld module) and can optionally include a frangible membrane 2455 (e.g., to maintain sterility).

The barrel 2440 includes an internal volume 2415. When a cap is present, then the cap also has an internal volume 2405 that is in fluidic communication with volume 2415. One or more structures can be present on a surface portion that defines the internal volume 2415 of the barrel 2440. Such structures include a locking member configured to affix the cartridge to the handheld module, as well as a sealing member to ensure a fluidic seal between the cartridge and the handheld module. An exemplary locking member includes a protrusion 2430 which is located on a surface portion defining the internal volume 2415, and this protrusion can either be a circumferential ring or a circular protrusion. An exemplary sealing member 2420 includes, e.g., an o-ring, located on a surface portion defining the internal volume 2415. A skilled artisan would understand how to place these structures to optimize locking and sealing, respectively.

The corresponding handheld module includes a mounting shaft that is configured to match the cartridge. As seen in FIG. 24, the mounting shaft 2460 is disposed on the distal section of the body and includes a central bore 2465 in fluidic communication with the cartridge. The mounting shaft is configured to be inserted into the opening 2450 of the disposable cartridge 2400 (e.g., any herein, including module forms thereof). The mounting shaft can include a fitting structure and/or a sealing structure disposed on an outer surface portion of the mounting shaft, where the fitting structure is configured to interface with the locking member of the disposable cartridge and where the sealing structure is configured to interface with the sealing member of the disposable cartridge. An exemplary fitting structure includes a notch 2461 that is configured to interface with the protrusion 2430 of the cartridge. An exemplary sealing structure includes a groove 2462 that is configured to interface with the sealing member 2420 of the cartridge. To facilitate release of the cartridge, the handheld module can optionally include one or more levers 2470 that eject the cartridge 2400 from the mounting shaft 2460.

FIG. 25A-25B provides another exemplary cartridge 2500 and its corresponding handheld module. The cartridge 2500 includes a barrel 2540 and a cap 2502 configured to house the substrate 2503 and microneedle array 2501. The cap 2502 is disposed at the distal end of the barrel 2540, and an opening 2550 is disposed at the proximal end of the barrel 2540. Again, the opening can optionally include a frangible membrane (e.g., to maintain sterility).

The barrel 2540 includes an internal volume 2505. One or more structures can be present on a surface portion that defines the internal volume 2505 of the barrel 2540. Here, the exemplary locking member includes a ridge 2530 which is located on a surface portion defining the internal volume 2505, and this ridge can either be disposed circumferentially or disposed in one particular cross-sectional location. The ridge can optionally also serve as a sealing member. As can be seen, the corresponding mounting shaft 2560 includes a central bore 2565 in fluidic communication with the cartridge, as well as a stop member 2566 that interfaces with the ridge 2530 of the cartridge.

The handheld module can include other structural attributes for the mounting shaft. For instance, as seen in FIG. 25B, the mounting shaft can include an additional sealing structure, such as a groove 2562 configured to align a sealing member 2561 (e.g., an o-ring) with a surface portion defining the internal volume 2505 of the cartridge. In another embodiment, the mounting shaft can include a locking member or a sealing member that is a ledge 2563, which interfaces with the lip 2545 of the cartridge 2500, thereby stabilizing the module interface and minimizing fluid leakage. Optionally, the handheld module can optionally include a release mechanism 2570 (e.g., one or more levers).

The handheld module and disposable cartridge can be employed in any useful manner. In one instance, the handheld module can be used to affix the cartridge to the desired site (e.g., a sample site for the subject). Then, the handheld module can be activated to either perform a measurement and/or to actuate a pumping mechanism. Finally, the cartridge can be released from the handheld module after either obtaining a measurement (e.g., detecting the presence or absence of one or more markers) or acquiring the sample.

Alternatively, obtaining a sample may take an extended period of time (e.g., more than 5 seconds, 10 seconds, 20 seconds, 30 seconds, 1 minute, etc.). In particular embodiments, such acquisition times can be optimized to ensure sufficient flow of the sample, while minimizing pain and discomfort to the subject. When extended acquisition times are needed, then the handheld module can be employed to apply the disposable cartridge and then detach the cartridge from the handheld module. In this way, the cartridge could collect sufficient fluid for analysis while the handheld module applies cartridge to other patients, thus gaining the ability to treat more patients. Once the cartridge has a suitable amount of fluid, the handheld module could be re-attached to the cartridge in order to perform a measurement.

Additional Components

The present device can include any useful additional component. Exemplary components include those provided for a transducer (e.g., any described herein, as well as those in Justino C I L et al., “Review of analytical figures of merit of sensors and biosensors in clinical applications,” Trends Analyt. Chem. 2010; 29:1172-83 and U.S. Pat. No. 6,398,931, each of which is incorporated by reference in its entirety); those provided for a microneedle (e.g., any described herein, as well as those in Gittard S D et al., “Two photon polymerization of microneedles for transdermal drug delivery,” Exp. Opin. Drug Deliv. 2010; 7(4):513-33, and Miller P R et al., “Multiplexed microneedle-based biosensor array for characterization of metabolic acidosis,” Talanta 2012; 88:739-42, each of which is incorporated by reference in its entirety); a membrane (e.g., placed between the needle and the channel; placed within a channel, such as to filter one or more particles within the sample; and/or placed between the channel and the electrode); a multifunctional sensor (e.g., to measure temperature, strain, and electrophysiological signals, such as by using amplified sensor electrodes that incorporate silicon metal oxide semiconductor field effect transistors (MOSFETs), a feedback resistor, and a sensor electrode in any useful design, such as a filamentary serpentine design); a microscale light-emitting diode (LEDs, such as for optical characterization of the test sample); an active/passive circuit element (e.g., such as transistors, diodes, and resistors); a release mechanism (e.g., as described in U.S. Pub. No. 2013/0306155, which is incorporated herein by reference in its entirety); an actuator; a wireless power coil; a device for radio frequency (RF) communications (e.g., such as high-frequency inductors, capacitors, oscillators, and antennae); a resistance-based temperature sensor; a photodetector; a photovoltaic cell; and a diode, such as any described in Kim D H et al., Science 2011; 333:838-43, which is incorporated herein by reference. These components can be made from any useful material, such as, e.g., silicon and gallium arsenide, in the form of filamentary serpentine nanoribbons, micromembranes, and/or nanomembranes.

The present device can include one or more structural components within the integral platform or substrate. Exemplary components include a mixing chamber in fluidic communication with the lumen of a needle; a reservoir optionally including one or more reagents (e.g., any described herein), where the reservoir can be in fluidic communication with the mixing chamber or any fluidic channel; a controllable valve (e.g., configured to release a reagent from a reservoir into a mixing chamber); a pump (e.g., configured to facilitate flow of a sample to the transducer and/or through one or more fluidic channels); a waste chamber (e.g., configured to store a sample after detection of one or more reagents); a probe; and/or a filter (e.g., configured to separate one or more components from the sample either before or after detection with the transducer).

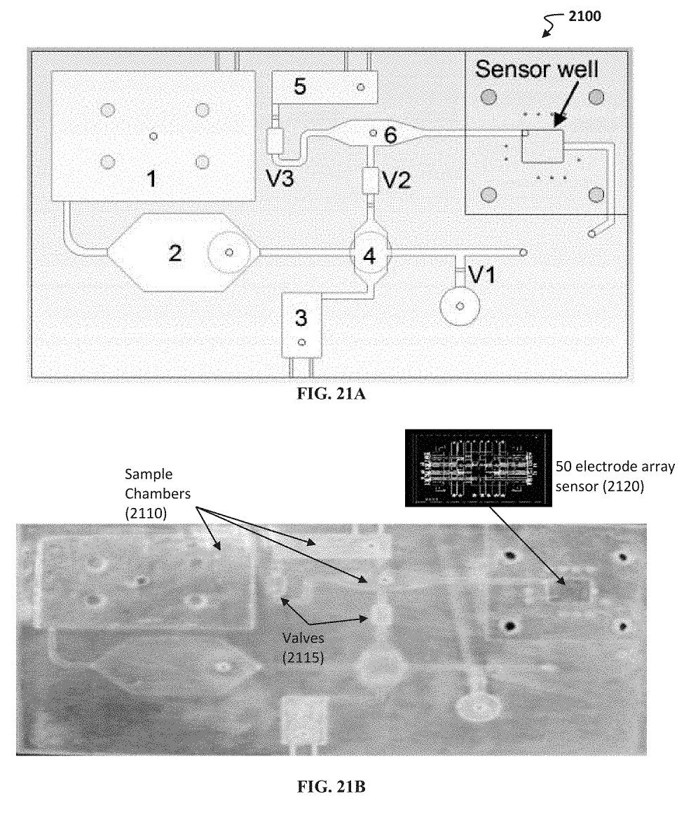

FIG. 21A-21B shows one non-limiting embodiment of structural features within a microfluidic modular package 2100 between the needle array and the detector. This package contains the mechanisms responsible for cell lysis and washing, and the extraction, cleavage, and movement of DNA.

As shown in FIG. 21A, chamber 1 contains a wash buffer. In one instance, the needle array (e.g., configured as a disposable cartridge) can be in fluidic communication with the sample chamber (e.g., chambers 1 or 5 in FIG. 21A or sample chambers 2110 in FIG. 21B). Chamber 2 contains one or more reagents to lyse cells collected from needles, as well as magnetic beads to bind DNA. After the lysis and wash from chamber 1, the magnet is removed, and the wash buffer flushes DNA into chamber 4, where another magnet recollects the beads. With the magnetic beads in chamber 4, valve one (marked V1) actuates and directs further flow through the rest of the reaction chambers. Elution buffer from chamber 3 removes DNA from the beads.

Actuation of valve two (V2 in FIG. 21A or one or more valves 2115 in FIG. 21B) forces the remaining sample into the final reaction chamber 6. There, the DNA alone is available to be cleaved by restriction enzymes already placed in the chamber. The remaining magnetic beads can be maintained in chamber 4 via reapplication of a magnetic field. After the DNA is digested, valve three (V3 in FIG. 21A or one or more valves 2115 in FIG. 21B) is actuated, and an EDTA (ethylenediaminetetraacetic acid) wash from chamber 5 deactivates the restriction enzyme. The EDTA wash is then used to push the contents of chamber 6 onto the microelectrode sensor array for analysis (e.g., a detector, such as a detector module, including any herein, such as an electrode array sensor 2120 in FIG. 21B). In a similar manner, the package can include one or more channels, chamber, and valves to perform any other useful marker.

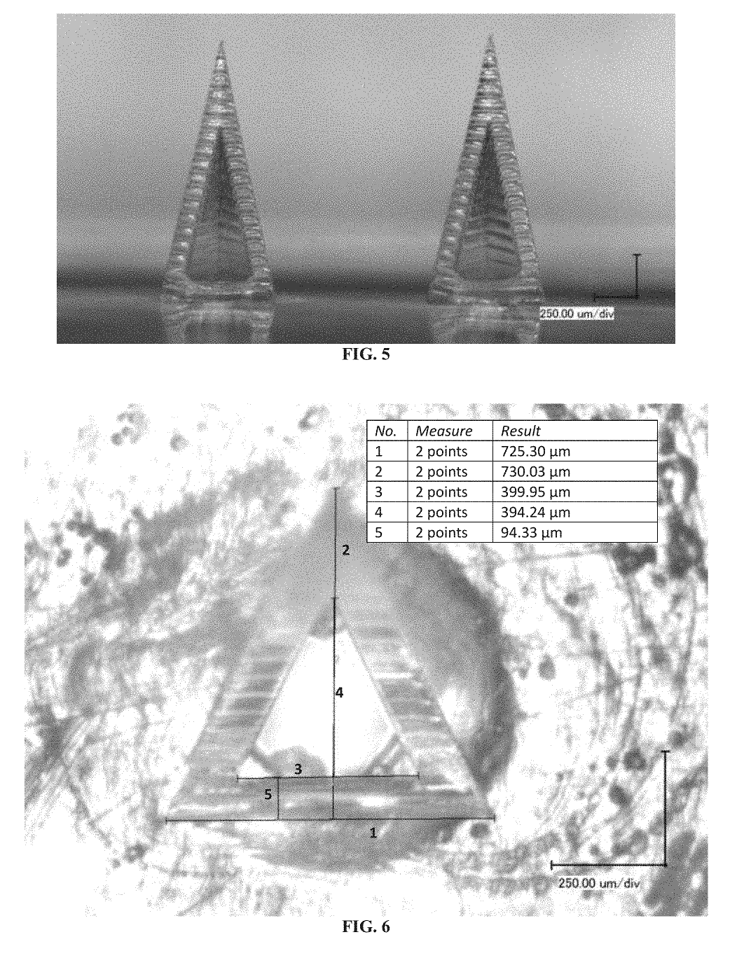

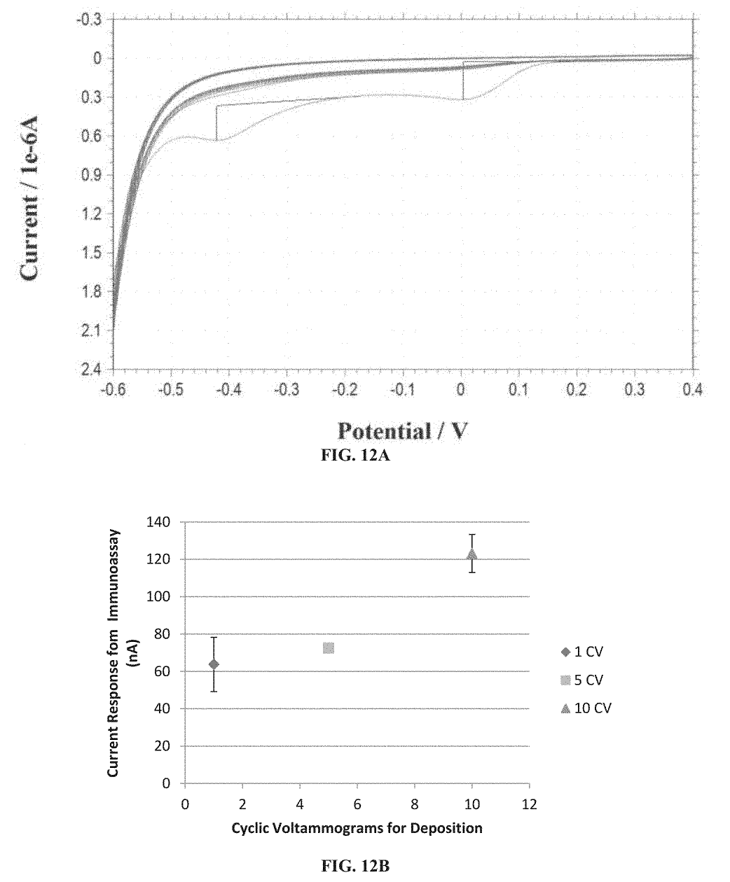

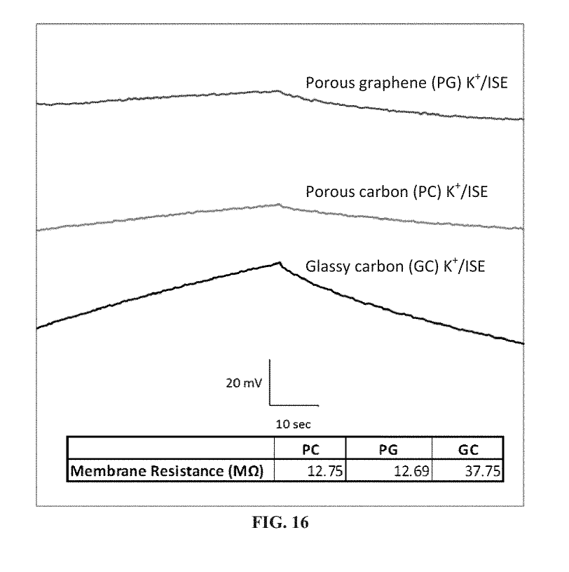

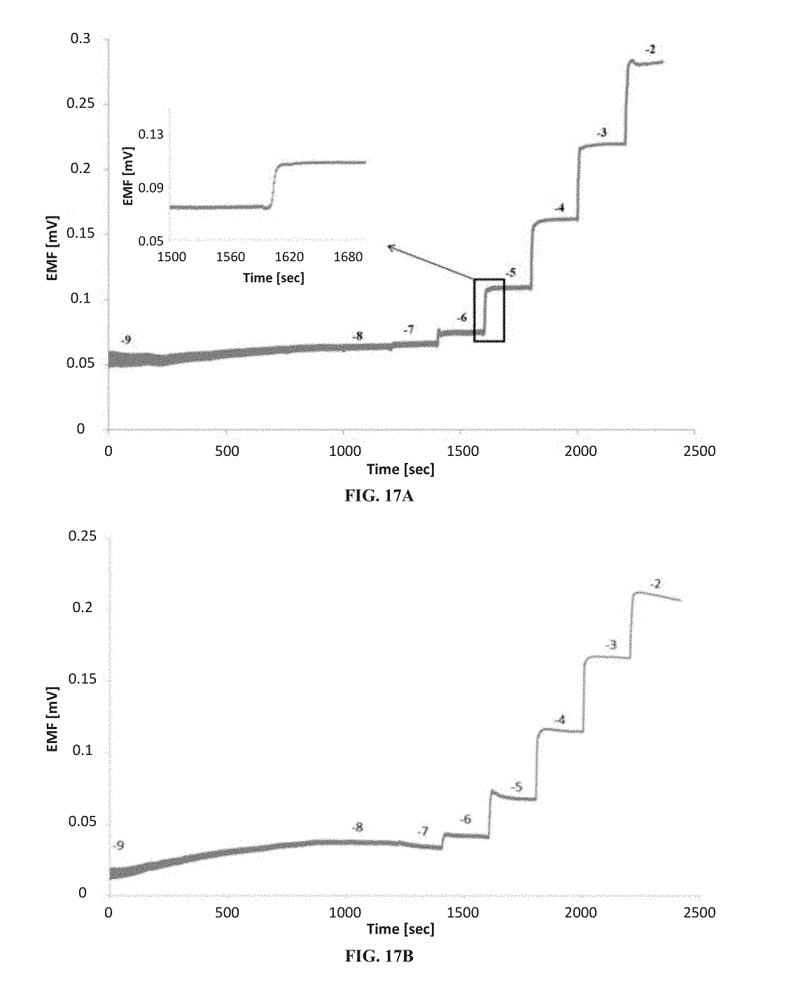

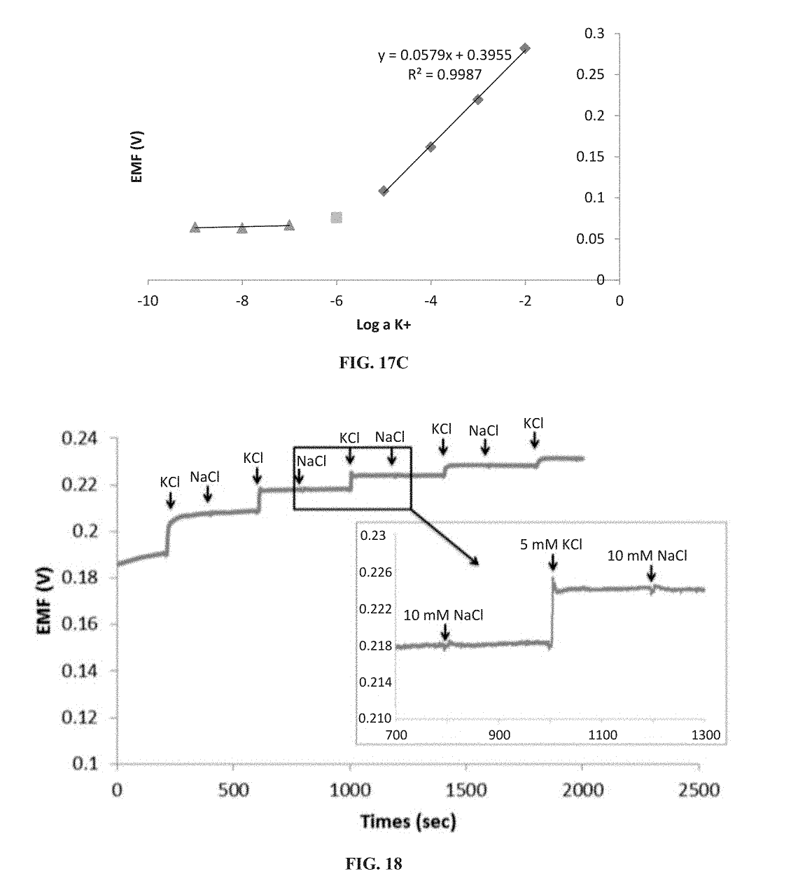

In some embodiments, the needle can be configured to be in fluidic communication with a reservoir (e.g., containing a drug for delivery and/or a reagent for detecting the marker of interest). Such a configuration can optionally include a valve between the needle and reservoir. In other embodiments, a probe can be configured to be in fluidic communication with the lumen of the needle. Exemplary needles and probes are described in Int. Pub. No. WO 2013/058879 (e.g., in FIG. 1A-1D, FIG. 1L, FIG. 2A-2C, FIG. 5A-5D, FIG. 12A-12B, FIG. 17, FIG. 18A-18D, and its related text), which is incorporated herein in its entirety.

The device can include one or more components to operate a transducer. For instance, in some embodiments, the transducer is an electrode or an array of electrodes. Accordingly, the device can further include a power source to operate the electrode. In particular embodiments, the device includes a data-processing circuit powered by the power source and electrically connected to the transducer (e.g., a counter electrode, a reference electrode, and at least one said working electrode). In further embodiments, the device includes a data output port for the data-processing circuit. Such data from the transducer can include any useful information, such as electromotive force (EMF), potentiometric, amperometric, impedance, and/or voltammetric measurements. Other data can include fluorometric, colorimetric, optical, acoustic, resonance, and/or thickness measurements.

The present invention can be useful for autonomous remote monitoring of a subject. The device of the invention can be placed on the skin of a subject, and the presence or absence of one or more markers can be remotely relayed to a heath care worker. Accordingly, the device described herein can include one or more components that would allow for such relay. Exemplary components include an analog-to-digital converter, a radiofrequency module, and/or a telemetry unit (e.g., configured to receive processed data from a data-processing circuit electrically connected to the transducer and to transmit the data wirelessly). In various embodiments, the telemetry unit is fixed within the platform or packaged separately from the platform and connected thereto by a cable.

Multiple Reactions

The present device can be used to perform multiple reactions on-chip. Such reactions can include those to prepare a sample (e.g., to dilute, concentrate, or filter a sample), to bind the sample to a capture agent, to prepare one or more reagents to be reacted with the sample (e.g., to reconstitute a reagent on-chip prior to reacting with the sample), to react the sample with any useful reagent, to store the sample on-chip, and/or to perform other post-processing reactions. To perform multiple reactions, the microneedles, fluidic channels, and transducers can be provided in an array format, such as any described herein.

To allow for multiple reactions or processing steps, the device can include additional chambers in fluidic communication with one or more needles. In one embodiment, the device include one or more mixing chambers in fluidic communication with one or more needles and configured to receive the sample or a portion thereof. The mixing chamber can include one or more reagents (e.g., any described herein), buffers, diluents (e.g., water or saline), salts, etc. Optionally, the mixing chamber can include one or more components to assist in mixing, such as one or more of the following: a bead, a passive mixer, a rotary mixer, a microbubble, an electric field to induce electrokinetic and/or dielectrophoretic flow, a staggered structure to induce chaotic advection, an acoustic mixer, a heater to induce a thermal gradient, and/or a magnetic bead for use with a magnetic field generator.

The device can also include one or more reaction chambers (e.g., to combine one or more reagents (e.g., one or more enzymes and/or beads) within this chamber and/or to incubate reaction mixtures including the sample or a portion thereof), lysing chambers (e.g., to lyse one or more cells within the sample), washing chambers (e.g., to wash one or more components within the sample), elution or extraction chambers (e.g., including one or more filters, particles, beads, sieves, or powders to extract one or more components from the sample), and/or collection chambers (e.g., to collect one or more processed samples or aliquots thereof). In particular embodiments, at least one reaction chamber is in fluidic communication with at least one mixing chamber by a channel. In further embodiments, the reaction chamber is in fluidic communication two or more mixing chambers, thereby combining the substance in each mixing chamber within the reaction chamber. In this manner, parallel or serial sequences of substances can be combined in a controlled manner within a reaction chamber or multiple reaction chambers. A skilled artisan would be able to design arrays of mixing and/or reaction chambers (optionally interconnected with channels) to effect the proper sequence of each reaction step.

Any of the chambers and channels interconnecting such chambers can be surface modified, as described herein. Furthermore, such chambers and channels can include further structures that would be useful for detecting one or more markers. For instance, one or more filters or membranes can be used to separate particular components from the sample and/or the reaction mixture. For instance, when the sample is whole blood, a filter can be used to separate the plasma from other blood components, such as the red blood cells.

Test Samples

The present device can be used to test any useful test sample, such as blood (e.g., whole blood), plasma, serum, transdermal fluid, interstitial fluid, sweat, intraocular fluid, vitreous humor, cerebrospinal fluid, extracellular fluid, lacrimal fluid, saliva, mucus, etc., and any other bodily fluid.

The sample can be obtained from any useful source, such as a subject (e.g., a human or non-human animal), a plant (e.g., an exudate or plant tissue, for any useful testing, such as for genomic and/or pathogen testing), an environment (e.g., a soil, air, and/or water sample), a chemical material, a biological material, or a manufactured product (e.g., such as a food or drug product).

Substances, including Reagents and Therapeutic Substances

The present device can further be adapted to deliver one or more substances from a reservoir to another region of the device or to a subject. In some embodiments, the device includes one or more reservoirs including a substance for detecting one or more markers of interest. Exemplary substances include a reagent (e.g., any described herein, such as a label, an antibody, a dye, a capture agent, etc.), a buffer, a diluent, a salt, etc.

In other embodiments, the device includes one or more substances that can be injected or delivered to a subject (e.g., one or more therapeutic substances). Such therapeutic substances include, e.g., an anesthetic, antiseptic, anticoagulant, drug, vaccine, etc.

Capture Agents

Any useful capture agents can be used in combination with the present invention. The capture agent can directly or indirectly bind the marker of interest. Further, multiple capture agents can be used to bind the marker and provide a detectable signal for such binding. For instance, multiple capture agents are used for a sandwich assay, which requires at least two capture agents and can optionally include a further capture agent that includes a label allowing for detection.

Exemplary capture agents include one or more of the following: a protein that binds to or detects one or more markers (e.g., an antibody or an enzyme), a globulin protein (e.g., bovine serum albumin), a peptide, a nucleotide, a nanoparticle, a microparticle, a sandwich assay reagent, a catalyst (e.g., that reacts with one or more markers), and/or an enzyme (e.g., that reacts with one or more markers, such as any described herein). The capture agent can optionally include one or more labels, e.g. any described herein. In particular embodiments, more than one capture agent, optionally with one or more linking agents, can be used to detect a marker of interest. Furthermore, a capture agent can be used in combination with a label (e.g., any described herein) to detect a maker.

Labels

The present device can include any useful label. The label can be used to directly or indirectly detect a marker. For direct detection, the label is conjugated to a capture agent that binds to the marker. For instance, the capture agent can be an antibody that binds the marker, and the label for direct detection is a nanoparticle attached to the capture agent. For indirect detection, the label is conjugated to a second capture agent that further binds to a first capture agent. For instance, as shown in FIG. 9, the label (HRP) is conjugated to a second capture agent (labeled “A”), where A furthers bind to a first capture agent (an antibody labeled with “B”). A skilled artisan would understand how to optimize combinations of labels, capture agents, and linking agents to detect a marker of interest.

Exemplary labels include one or more fluorescent labels, colorimetric labels, quantum dots, nanoparticles, microparticles, barcodes, radio labels (e.g., RF labels or barcodes), avidin, biotin, tags, dyes, an enzyme that can optionally include one or more linking agents and/or one or more dyes, as well as combinations thereof etc.

Markers, including Targets

The present device can be used to determine any useful marker or targets. Exemplary markers include one or more physiologically relevant markers, such as glucose, lactate, pH, a protein (e.g., myoglobin, troponin, insulin, or C-reactive protein), a catecholamine (e.g., dopamine, epinephrine, or norepinephrine), a cytokine (e.g., TNF-α, IL-12, or IL-1β), a biomolecule (e.g., cholesterol or glucose), a neurotransmitter (e.g., acetylcholine, glutamate, dopamine, epinephrine, or norepinephrine), a signaling molecule (e.g., nitric oxide), an electrolyte (e.g., potassium, sodium, chloride, bicarbonate, etc.), an ion (e.g., a cation, such as K⁺, Na⁺, H⁺, or Ca²⁺, or an anion, such as Cl⁻ or HCO₃⁻), CO₂, O₂, H₂O₂, a cancer biomarker (e.g., human ferritin, carcinoembryonic antigen (CEA), prostate serum antigen, human chorionic gonadotropin (hCG), diphtheria antigen, or C-reactive protein (CRP)), a hormone (e.g., hCG, epinephrine, cortisol, or a peptide hormone), an inflammatory marker (e.g., CRP), a disease-state marker (e.g., glycated hemoglobin for diabetes), a cardiovascular marker (e.g., CRP, D-dimer, troponin I or T), a viral marker (e.g., a marker for human immunodeficiency virus, hepatitis, influenza, Ebolavirus, or chlamydia), a metabolite (e.g., glucose, cholesterol, triglyceride, creatinine, lactate, ammonia, ascorbic acid, or urea), a nucleic acid (e.g., DNA and/or RNA for detecting one or more alleles, pathogens, single nucleotide polymorphisms, mutations, etc.), a drug (e.g., a diuretic, a steroid, a growth hormone, a stimulant, a narcotic, an opiate, etc.), etc. Other exemplary markers include one or more pathogens, such as Mycobacterium tuberculosis, Diphtheria antigen, Vibrio cholera, Streptococcus (e.g., group A), etc.

Methods and Use

The present device can be applied for any useful method and/or adapted for any particular use. For instance, point-of-care (POC) diagnostics allow for portable and/or disposable systems, and the device herein can be adapted for POC use. In some embodiments, the device for POC use includes a test sample chamber, a microfluidic processing structure (e.g., any structure described herein, such as a needle, a substrate, and/or a channel), a target recognition region (e.g., including any transducer described herein), an electronic output, a control (e.g., a positive and/or negative controls), and/or a signal transduction region. Exemplary POC devices and uses are described in Gubala V et al., “Point of care diagnostics: status and future,” Anal. Chem. 2012; 84(2):487-515, which is incorporated by reference in its entirety. Such POC devices can be useful for detecting one or more markers for patient care, drug and food safety, pathogen detection, diagnostics, infectious disease control (e.g., of any infection disease, such as a viral infection), etc. In some embodiments, the device of the invention is configured to monitor and/or detect signs and symptoms related to any infection (e.g., a pathogen or viral infection). Such signs and symptoms include, e.g., those related to hemorrhagic fever (e.g., arising from a viral infection from, e.g., an RNA virus, such as those in the following families: Arenaviridae (e.g., Lujo virus, Lassa virus, Junin virus, Machupo virus, Sabia virus, or Guanarito virus); Bunyaviridae (e.g., Hantavirus, Crimean-Congo hemorrhagic fever virus, or the Rift Valley fever virus); Filoviridae (e.g., Ebolavirus (including species of Zaire ebolavirus, Sudan ebolavirus, Taï Forest ebolavirus, and Bundibugyo ebolavirus) and Marburgvirus (Marburg marburgvirus)); or Flaviviridae (e.g., Yellow Fever virus, West Nile virus, Dengue Fever virus, Omsk hemorrhagic fever virus, or Kyasanur Forest disease virus)).

Wearable sensors are a new paradigm in POC devices, allowing for minimally invasive monitoring of physiological functions and elimination of biological fluid transfer between subject and device; these devices can be capable of providing real-time analysis of a patient's condition. In other embodiments, the device is adapted to include one or more components allowing for a wearable sensor. Exemplary wearable sensors, as well as relevant components, are described in Windmiller J R et al., “Wearable electrochemical sensors and biosensors: A review,” Electroanalysis 2013; 25:29-46. Such components include a telemetry network including one or more devices (e.g., as described herein), one or more flexible substrates (e.g., where one or more transducers are integrated into a flexible substrate, such as cloth, plastic, or fabric, e.g., Gore-Tex®, an expanded polytetrafluoroethylene (ePTFE), polyimide, polyethylene naphthalate, polyethylene terephthalate, biaxially-oriented polyethylene terephthalate (e.g., Mylar®), or PTFE), and/or one or more flexible electrodes (e.g., a screen printed electrode printed on a flexible substrate, such as any herein).

In some embodiments, the device of the invention is adapted as an epidermal electronic device. Such devices can include, e.g., one or more printed flexible circuits that can be stretched and bent to mimic skin elasticity can perform electrophysiological measurements such as measuring temperature and hydration as well as monitoring electrical signals from brain and muscle activity. Exemplary components for such a device are described in Kim D H et al., Science 2011; 333:838-43, which is incorporated herein by reference.

In other embodiments, the device of the invention is adapted as a disposable cartridge. Such devices can include one or more microneedles (e.g., an array of microneedles) disposed on the tip of a barrel, as described herein (see, e.g., FIGS. 22, 23, 24, and 25A-25B). The disposable cartridge can have a matching configuration to a mounting shaft, which is located at the end of a detector (e.g., an electronic controller and display capable of detecting one or more markers and relaying the results of the analysis to the user).

In yet other embodiments, the device of the invention is adapted as a temporary tattoo. Such tattoos can include, e.g., one or more screen printed electrodes directly attached to the skin were recently reported to measure lactate through sweat. Exemplary components for such a device are described Jia W et al., “Electrochemical tattoo biosensors for real-time noninvasive lactate monitoring in human perspiration,” Anal. Chem. 2013; 85:6553-60, which is incorporated herein by reference.

The device of the invention can be configured for any useful method or treatment. For instance, the device can be configured for locally treating, delivering, or administering a therapeutic substance after detecting one or more markers. Exemplary methods and devices are described in Int. Pub. No. WO 2010/022252, which is incorporated herein by reference.

Kits

The present device can be provided in any useful form, such as in a kit. In some embodiments, the device is provided in combination with an adhesive layer and a backing liner, where peeling of the backing liner exposes the adhesive layer and allows for positioning the device on the skin of a subject. In other embodiments, the kit includes a device (e.g., any described herein), an instruction for use, and, optionally, one or more therapeutic substances (e.g., any described herein).

Packaged Chip

The present device can be provided in any useful package. For instance, such a package can include a packaged chip having a housing for the device of the invention. In one embodiment, the housing includes a substantially planar substrate having an upper surface and an opposing lower surface; a first fluidic opening disposed on the upper surface of the substrate; a second fluidic opening disposed on the lower surface of the substrate; a first fluidic channel fluidically connecting the first fluidic opening to the second fluidic opening; and a first adhesive layer adhered to the upper surface, having a hole disposed through the layer, wherein the hole is substantially aligned with, and fluidically coupled to, the first fluidic opening in the substrate. In some embodiments, the housing includes one or more structures allowing for integrating with a fluidic printed wiring board having a standard electrical printed circuit board and one or more fluidic channels embedded inside the board. An exemplary packaged chip is provided in FIG. 11 and associated text describing this figure in U.S. Pat. No. 6,548,895, which is incorporated by reference in its entirety. Further components for a packaged chip include a substrate including an electrically insulating material, one or more electrical leads, a substantially planar base, an external fixture, etc., as well as any other components described in U.S. Pat. Nos. 6,443,179 and 6,548,895, each of which is incorporated herein by reference in its entirety.

The device of the invention can be provided in any useful format. For instance, the device can be provided with particular components integrated into one package or monolithic structure. A non-limiting example of such an integrated device is provided in FIG. 1, where the needles, fluidics, and electrode array are provided in an integrated format. In other examples, the device is provided as a modular package, in which the needles, fluidics, and electrodes are provided as separate plug-and-play modules that can be combined. A non-limiting example of such a modular package is provided in FIG. 21A-21B.

In particular embodiments, a sensor module includes a packet of electrode array with each packet containing specific chemistries. In further embodiments, the sensor module is configured to be relevant for the desired analyte, such as to detect a particular drug or a particular virus. Further modules can include a needle module including one or more needles (e.g., an array of needles); a fluidics module including one or more chambers, valves, and/or channels; and/or a reagent module including one or more prepackaged reagents and buffers configured for a particular test or analyte.

Such modules can be reusable or disposable. For instance, if the sample processing is extensive, one would want a reusable fluidics module, which is configured for fluidic communication with the needle module and sensor module. In further embodiments, the needle and sensor modules can be disposable. In another example, if sample processing or sensing requires an elaborate needle (e.g., a needle having a particular geometrical configuration and/or surface modification), then the needle module can be configured to be reusable. Other considerations include possibility of contamination of one or more modules, etc. A skilled artisan would understand how modules can be configured for fluidic communication with other modules and designed for reusability or disposability.

### EXAMPLES

### Example 1

### Integrated Needle/Transducer Microfluidic Manifold

An integrated device was constructed and tested. A hollow needle manifold was developed complete with integrated electrode arrays and a fluidic channel. The resulting chip was packaged using plastic laminate technology with a fluidic channel that could access the needles and flow solution over the electrode array. This type of device is a significant advancement towards an autonomous needle platform, which is capable of transdermally accessing interstitial fluid and performing real time and repeated measurements for a variety of physiologically relevant analytes. A description of this device follows.

Materials and Methods

Cleaning of Au Arrays: Gold electrode arrays were cleaned to remove residual material from the fabrication process since electrodes coming directly from the final fabrication step were not suitable for analytical electrochemical studies. Cleaning was done with a combination of a chemical treatment and an electrochemical treatment. First, gold electrode arrays were sonicated in a solution of 50 mM KOH and 25% H₂O₂ for 10 minutes with solutions that were made fresh daily. One cyclic voltammogram (CV) was used as the electrochemical treatment in 50 mM KOH; scanning from −200 mV to −1200 mV at 50 mV/s against an Ag/AgCl reference electrode and a platinum wire counter electrode was performed. Electrodes were then washed with isopropanol, washed with DI H₂O, and dried with a nitrogen stream. For electrodes that used Melinex® (polyester) windows, which were defined by laser cutting, the windows were applied after the cleaning steps.

Preparation of Carboxyl Diazonium: Modification to the gold array surfaces for attachment of the primary antibody was done by depositing a carboxyl diazonium with cyclic voltammograms. The carboxyl diazonium molecules were created in-situ by combining 10 mM aminophenyl propionic acid and 8 mM sodium nitrite in 0.5 M HCl for 10 minutes; this step was performed in the dark. The solution was then immediately deposited on cleaned gold arrays by running cyclic voltammograms from 0.4 V to −0.6 V at 100 mV/s against an Ag/AgCl reference electrode and a platinum wire counter electrode. Once deposited, electrodes were cleaned by sonicating in DI H₂O for 30 seconds, washing with DI H₂O, and then drying with nitrogen.

Immunoassay Procedure: Gold arrays with deposited carboxyl diazoniums were treated with EDC/NHS chemistry to activate the COOH group in order to attach the primary antibody. A solution of 100 mM EDC and 25 mM NHS in 10 mM HEPES buffer (pH=7.4) was applied to the surface of the gold arrays and left for 30 minutes. The solution was then washed off with 10 mM HEPES (pH=7.4). Two ppm of the primary antibody in 10 mM HEPES (pH=7.4) was applied to the surfaces of the electrodes. The appropriate primary antibodies for detection of myoglobin or troponin ITC complex were used in this study. The electrodes were again rinsed with 10 mM HEPES buffer (pH=7.4) and incubated in 1% BSA in 1× PBS (pH=7.2) for 30 minutes to block unbound active sites. The electrodes were thoroughly washed with 1× PBS (pH=7.2) and then treated with the desired concentration of protein in 10 mM HEPES solution (pH=7.4) for 1 hour. The electrodes were subsequently washed with 1× PBS (pH=7.2). A 2 ppm solution of the secondary antibody in 10 mM HEPES solution (pH=7.4) was applied to the gold arrays, incubated for 1 hour, and washed with 1× PBS (pH=7.2). Following thorough washing of secondary-treated gold arrays, electrochemical detection was performed.

TMB conductivity solution was applied to the electrodes and a chronoamperometric scan was run at 0 V for 30 seconds against an Ag/AgCl reference electrode and a platinum wire counter electrode. For tests done to determine the optimal number of cyclic voltammograms for the diazonium deposition, the secondary antibody was directly attached to the activated COOH-terminated diazonium and then tested against the conductivity solution.

Results and Discussion

Fabrication of Hollow Microneedles: Hollow microneedles were made using a laser direct write system that utilizes the two photon polymerization approach. First, substrates for the microneedles were made by molding a piece of PMMA (2 mm×10 mm×10 mm) with PDMS, which was allowed to cure overnight. 250 μl of Eshell 300, an acrylate-based material that is used to manufacture hearing aid shells, was poured into the mold and polymerized with a UV lamp for 20 minutes. Bores were created in the Eshell 300 substrates to create a fluidic path between the hollow microneedles and the microfluidic chip. The bores were prepared by writing a 150 μm circle into the substrate with a CO₂ laser cutter. The exit bore was measured with an optical microscope to ensure that bores with an appropriate size were created; bores between 100 μm and 150 μm were considered to be suitable. Substrates were then washed with isopropanol to remove residual ablated material.

Microneedle fabrication was performed by creating a reservoir around the bore of the Eshell 300 substrate with a parafilm spacer. The well was filled with Eshell 300; a glass coverslip was placed on top, minimizing inclusion of bubbles within the polymerization cell. A vacuum was pulled briefly at the backside of the substrate to introduce a small amount of resin into the bore. This approach enabled a portion of the microneedle to be written within the bore, which improved the strength of the needle/substrate interface and removed air bubbles around the substrate bore. The hollow microneedles were designed in Solidworks 3D design software (Dassault Systémes, S. A., Vélizy, France) and the STL files were then read using GOLD3D custom laser direct software (Newport Spectra, Newport, Calif.) (see, e.g., FIG. 4 for an exemplary microneedle). Completed microneedles were developed in isopropanol for 5 minutes. A vacuum was again pulled at the backside of the substrate to ensure that the bores were free of residual resin. Hollow microneedles were post-cured under a UV lamp to ensure complete polymerization.

The light source for the fabrication of the hollow microneedles was a Ti:sapphire laser that was operated at 800 nm wavelength, 150 fs pulse length, and 80 MHz repetition rate. The beam of the laser was focused onto the sample with a 4× objective to increase the photon density and obtain two photon polymerization of the resin.

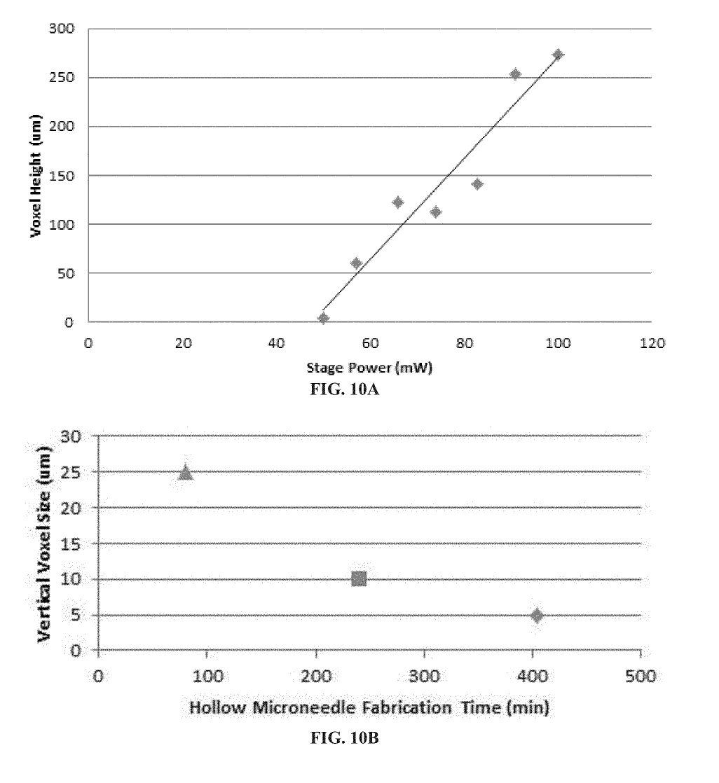

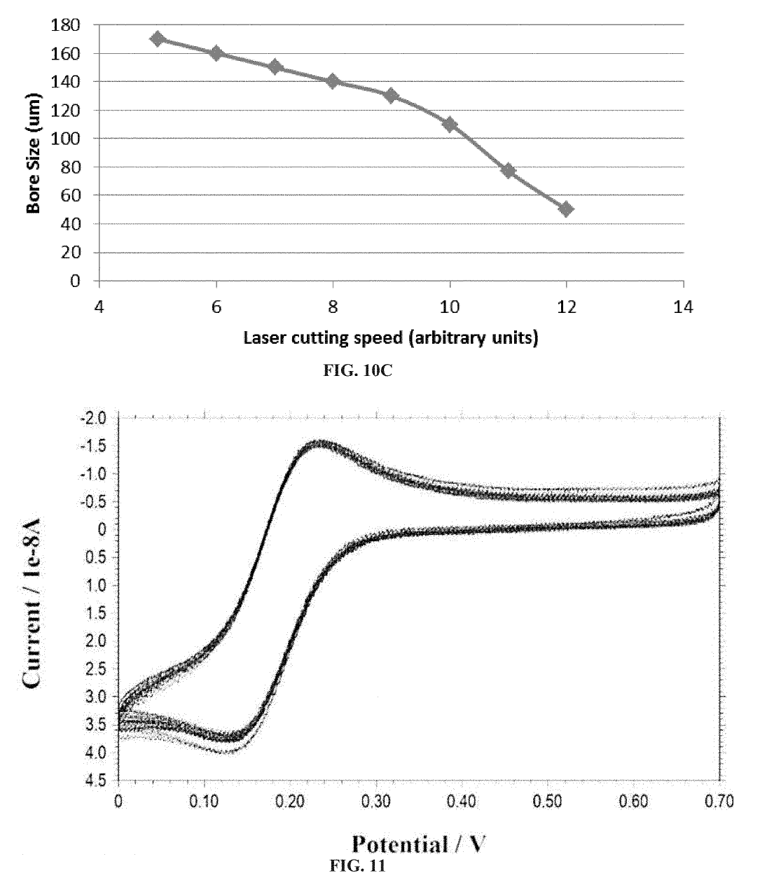

Prior to microneedle fabrication, characterization of the two photon polymerization process for the Eshell 300 resin and the objective was determined. FIG. 10A shows the length of the vertical voxel created at each tested energy. These results were used to optimize the step height between each layer, which affects the fabrication time for the microneedle. FIG. 10B shows fabrication times for the same hollow microneedle with dimensions of 500 μm by 1000 μm, in which only the spacing between each layer was altered. Parameters for microneedle fabrication that were used in this study include a step height of 25 μm and a laser energy below 60 mW, due to the fact that laser power values above 60 mW were associated with over-polymerization and clogged hollow microneedle bores.

Electrode Array Fabrication and Characterization: An 8 element electrode array for integration with the microneedle array is presented in FIG. 8. Six inch diameter glass wafers were utilized as substrates for the electrode arrays. Standard photolithography techniques were used to pattern 150 Å Cr/3000 Å Au electrodes and contact pads. In order to precisely define the electrode surface area, a 2000 Å thick silicon nitride layer was deposited at 350° C. over the entire device using PECVD. A photolithography step defined a precise opening over the dielectric layer, which measured 112 μm wide by 150 μm high. An SF₆ plasma etch was then used to selectively remove the exposed silicon nitride until the Au layer underneath was reached. The magnified image in FIG. 8 shows the 1120 μm×150 μm Au working area, which is the only part of the electrode that was exposed to solution and is electrochemically active. Also shown are the counter and reference electrodes, which were patterned on the chip. The devices were then cut using a dicing saw. The final process step was a 40 minute oxygen plasma cleaning step for stripping fluorocarbon and photoresist residue from the chip surface.

Potential cycling in potassium ferricyanide mediator was then performed to assess electrode reproducibility and quality (FIG. 11). Overlays of the responses of the electrodes in ferricyanide solution show ΔEp values of ˜60 mV, with approximately 20 nA variations across the 8 element electrode array. This result indicates that the electrode response was highly reproducible and suitable for electrochemical measurements. The quasi-sigmoidal character of the voltammograms is indicative of the small working area of each electrode, resulting in hemispherical diffusion responses. Hemispherical diffusion is well known to be associated with high signal to noise responses.

Electrode Array Optimization for Immunoassay: The electrode array was optimized for immunoassays to detect either troponin or myoglobin. Troponin and myoglobin were utilized in the clinical setting as biomarkers for detection of cardiac and skeletal muscle injuries, respectively. The approach used (see, e.g., Polsky R et al., “Electrically addressable diazonium-functionalized antibodies for multianalyte electrochemical sensor applications,” Biosens. Bioelec. 2008; 23:757-64, which is incorporated herein by reference in its entirety) in this study was a sandwich antibody assay, consisting of a capture antibody and a secondary detection antibody labeled with a horseradish peroxidase enzyme that catalyzes conversion of a TMB substrate to an electrochemically-detectable product (FIG. 9). The electrodes were first modified with phenyl molecules to immobilize capture antibodies by electrochemical reduction from the corresponding phenyl diazonium molecules (FIG. 9 inset) (Polsky R et al., “Multifunctional electrode arrays: Towards a universal detection platform,” Electroanalysis 2008; 20:671-9, which is incorporated herein by reference in its entirety). This procedure is described in detail in the experimental section.

Characterization of Diazonium Deposition: Electrochemical deposition of carboxyl diazonium on gold electrodes resulted in an irreversible reduction wave on the first scan of a cyclic voltammogram shown in FIG. 12A. Reduction peaks at ˜0 V and ˜400 mV are indicative of the in-situ generated diazonium reduction at different crystal planes on the gold electrodes. Subsequent voltammograms showed no reduction waves due to electrode passivation from phenyl radical grafting, a process that is commonly observed during deposition of phenyl diazonium molecules.

Optimization of carboxyl diazonium deposition parameters was performed to determine the number of cyclic voltammograms necessary for the largest and most consistent current responses from directly conjugating the HRP-labeled antibody and measuring enzymatic activity by means of electrochemical transduction. Cleaned electrodes were deposited with the in-situ carboxyl diazonium at 1, 5, or 10 cyclic voltammograms. The secondary antibody was then attached via EDC/NHS chemistry and tested against the TMB conductivity solution. Electrodes with 1, 5 and 10 cyclic voltammograms of diazonium deposition exhibited an average current response of 63 nA, 74 nA and 123 nA, respectively (FIG. 12B). Electrodes that were modified using 10 cyclic voltammograms of diazonium deposition produced the largest magnitude and most consistent reduction signals; therefore, electrodes modified using 10 cyclic voltammograms were utilized for all further depositions.

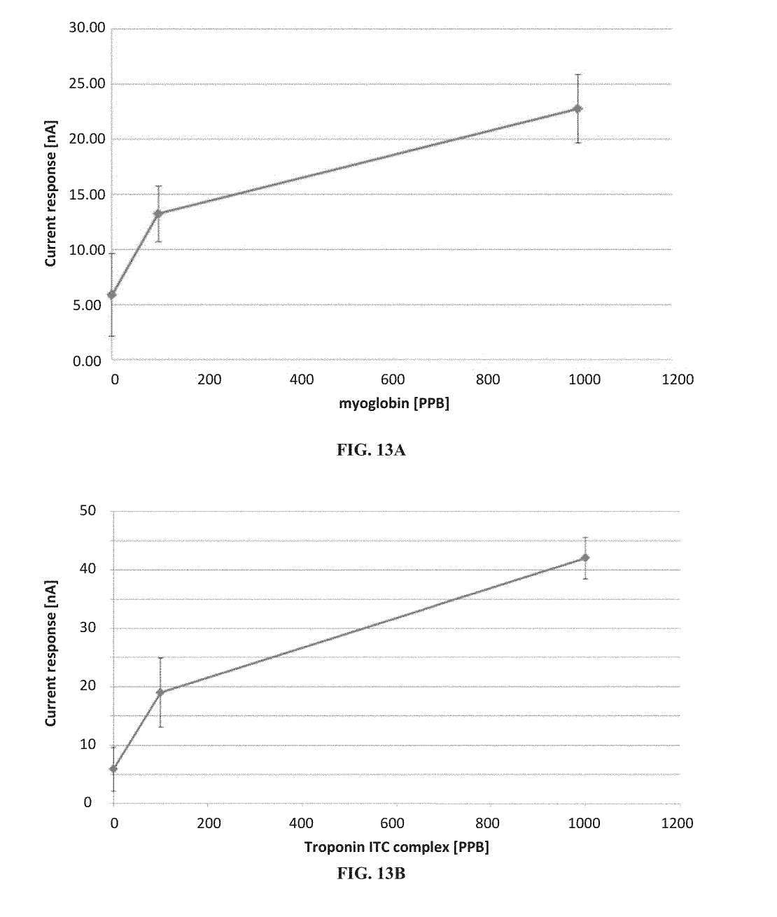

Immunoassay characterization: Sandwich immunoassays consisted of exposure to the target protein followed by HRP-labeled capture antibody treatment (see above). A fast steady state current was achieved upon chronoamperometric biasing of the electrode to 0 V, corresponding to the electroreduction of the oxidized TMB mediator. After exposure to varying protein concentrations, the sensor response was obtained 5 s after the potential step. Calibration curves generated for myoglobin and troponin ITC complex are presented in FIG. 13A and FIG. 13B, respectively. A dependence of signal on concentration was observed for both proteins between 100 ppb and 1000 ppb, demonstrating that this technique is suitable for quantitative detection.

Integration of Microneedles and Fluidic Chip: A microfluidic manifold was constructed from acrylic sheets and medical grade PSA using a precision cutting laser. The acrylic sheets were typically 2 mm in thickness, though thinner or thicker sheets could be used depending on application, mechanical robustness associated with intended function, and form factor. Medical grade PSA was chosen because it is frequently used in the construction of commercial bioassay devices; it has demonstrated low outgassing, low chemical leaching, and biocompatibility. Each of these materials was cut with a laser and sequentially assembled on a jig to create complex fluidic networks, with lateral flow channels being formed in the adhesive layers and connecting vias being formed in the acrylic sheet. Once the layers are stacked and assembled, they are pressed together for 2 minutes at 500 psi to assure good adhesion of the laminate layers. Our design features one acrylic layer and two adhesive layers, with the bottom adhesive layer forming the flow channel on the surface of the electrochemistry sensing chip and the top adhesive layer sealing the microneedles to the laminate cartridge. In order to provide for external fluid connections, we enclosed conventional Viton rubber O-rings (size 001) within the cartridge. Inserting 1/32″ tubing into these captured O-rings will create a fluid-tight seal and will allow pressure to either inject or draw fluid through the microneedle array. The final integrated package is shown in FIG. 1.

Conclusions: In conclusion, we have developed a hollow microneedle manifold complete with integrated electrode arrays and a fluidic channel. An 8 channel electrode array was fabricated using photolithographic patterning and dielectric insulating layers to expose a 112 μm wide by 150 μm Au working area, which was used as an electrochemical transducer. Potassium ferricyanide cycling was used to characterize the response over the 8 working electrodes, which were shown to be highly reproducible and suitable for electrochemical measurements. The electrodes were then modified using potentially addressable diazonium chemistry to immobilize a capture antibody. The electrodes were subsequently optimized for the detection of target proteins troponin, a cardiac injury marker, and myoglobin, a skeletal injury marker, using an electrochemical sandwich immunoassay protocol. The resulting chip was packaged using plastic laminate technology with a fluidic channel that could access the microneedles and flow solution over the electrode array. This type of device is a significant advancement towards an autonomous microneedle platform, which is capable of transdermally accessing interstitial fluid and performing real time and repeated measurements for a variety of physiologically relevant analytes.

### Example 2

### Microneedle-Based Transdermal Sensor for On-chip Potentiometric Determination of IC

The integrated device of the invention can include any other useful components. For instance, the detection of an ion can include use of an ion-selective electrode. A non-limiting description of such a device follows.

The development of a transdermal sensing device capable of measuring potassium with a solid-state ion-selective-electrode (ISE) by integrating a hollow with a microfluidic chip is described. Porous carbon and porous graphene electrodes, made via interference lithography, were investigated as transducers for ISE's in terms of their electrochemical performance, stability, and selectivity. The porous carbon K⁺ ISE's showed better performance compared to the porous graphene K⁺ ISE's and were capable of measuring potassium across a range concentrations, showed suitable performance against interfering ions found in physiological samples, and had a comparable degree of stability. A new method for incorporating hollow microneedles into a microfluidic chip was created and shown and may have applications to other devices. The device, as described below, was shown to detect potassium on-chip across a range of physiologically normal and abnormal values.

Microneedle-enabled analysis systems are capable of minimally-invasive interrogation due to their ability to puncture the skin's stratum corneum and access interstitial fluid while not interacting with deeper layers of the skin, which contains tissues that are associated with pain, blood flow, or sensation (see, e.g., Kim Y et al., Adv. Drug Deliv. Rev. 2012; 64:1547-68; and El-Laboudi A et al., Diabetes Technol. Therap. 2013; 15:101-15). For example, glass microneedle arrays were used to create pores in the skin in order to extract interstitial fluid by means of a vacuum bell jar for glucose detection with commercially available glucose strips (Ping J et al., Electrochem. Commun. 2011; 13:1529-32). A strong correlation was shown between intravenous glucose concentrations and those within dermal tissue. In another study, the surfaces of solid gold microneedles were functionalized with antibodies, which were used to collect nonstructural protein-1 (an early marker for dengue virus infection) in mice. When inserted in the skin, the functionalized microneedles bound the protein and remained attached; once the needles were removed from the animal, further ex vivo analysis was performed (Muller D A et al., Anal. Chem. 2012; 84:3262-3268). Several examples involving microneedles and electrochemical detection have also been recently reported, including packing of hollow microneedles with enzyme filled carbon pastes to amperometrically detect glucose or glutamate (Windmiller J R et al., “Bicomponent microneedle array biosensor for minimally-invasive glutamate monitoring,” Electroanalysis 2011; 23:2302-9), and use of a multiplexed microneedle device to simultaneously measure glucose, lactate, and pH (Miller P R et al., Talanta 2012; 88:739-42).

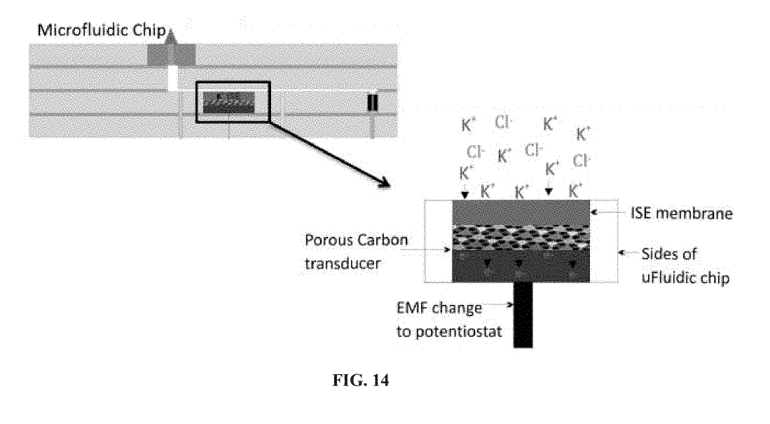

Electrolytes are important for maintaining cell signaling, kidney function, homeostasis, and body fluid balance. Electrolyte levels can fluctuate due to exercise, diet, disease, poisoning, and organ failure, making their monitoring invaluable for healthcare assessment. Solid state ion selective electrodes (ISE) that incorporate H⁺ ionophores and solvent polymeric membranes have been shown to be efficacious for metal cation determination (Bakker E et al., Chem. Rev. 1997; 97:3083-132). In contrast to liquid based ISEs, solid state ISEs require less maintenance and compatible with microfabrication and array construction methods. Buhlmann and Stein introduced the use of three-dimensional macroporous carbon electrodes, which were prepared from colloidal sphere templating (3DOM), as a solid contact for PVC-doped valinomycin sensing membrane-based K⁺ detection (Lai C et al., Anal. Chem. 2007; 79:4621-26). The highly ordered three-dimensional porous carbon structures provided high capacitance and a large interfacial area, which resulted in excellent performance and long term stability of the sensor compared to non-porous carbon analogues. Here, we describe the use of interferometric lithographically-fabricated three dimensional porous carbon electrodes integrated into a microfluidic channel for the construction of a K⁺ microneedle sensor. FIG. 14 is a schematic showing EMF generation at such an electrode. Details are provided herein.

Experimental

Porous carbon fabrication: Porous carbon substrates were prepared using an interference lithography method. In this method, negative tone NR-7 coated substrates were exposed to a frequency-tripled 355 nm line of Q-switched Nd:YAG laser. The laser beam was expanded and split so it could be interfered with at 32 degrees between the planewave propagation vectors. The plane of incidence contained both propagation vectors as well as the angle bisector of the propagation vectors, which was tilted with respect to the sample surface normal by 45 degrees.

Creation of the porous architecture was achieved by rotating the sample 120 degrees following each exposure and repeating the process three times to ensure proper exposure dosages. Silicon substrates were prepared by spinning an anti-reflecting coating of iCON-7 (Brewer Science, Rolla, Mo.) at 3000 rpm, followed by baking on a vacuum hotplate at 205° C. for 60 s. An adhesion layer was created by spinning NR7 100 P (˜100 nm) at 3000 rpm; this layer was flood exposed and baked at 130° C. on a vacuum hotplate. The layer to be patterned was subsequently spun; NR7-6000 P was spun at 3000 rpm (6 μm) and soft-baked at 130° C. Following the patterning exposure, substrates were baked at 85° C. for 2 minutes on a vacuum hot plate and then puddle developed for 120 s using RD-6 (Futurrex, Inc.). Spinning drying was used to remove any residual developer from the structures. Substrates were then baked on a hotplate at 180° C. for 30 minutes, and pyrolyzed at 1100° C. for 1 hour.

Electrode preparation: Porous carbon (PC) electrodes were cut into ˜8 mm×˜14 mm pieces from bulk Si substrates with a diamond scribe. This electrode size was consistent throughout the experiments; eight pieces were obtained from one bulk Si wafer. PC substrates were cleaned by washing with isopropanol and DI H₂O; drying with nitrogen was subsequently performed.

Potassium selective membranes were prepared by creating a cocktail solution by mixing 1% valinomycin, 0.3% KTFPB (potassium tetrakis[3,5-bis(trifluoromethyl)phenyl]borate), 66% o-NPOE (o-nitrophenyloctyl ether), and 32.8% PVC (poly(vinyl) chloride) in THF at 15 wt/%. PC electrodes were coated by pipetting 100 μl of the ISE cocktail onto the substrates; this material was allowed to dry for 24 hours. Conditioning of the PC/K+ISE's was performed overnight in 10 mM KCl. The electrodes were stored in this solution when not in use. A planar carbon electrode and a 3 mm carbon disc (BASi) electrode were used in this study. The planar carbon electrodes were prepared by first hand polishing with 0.3 μm and 0.05 μm alumina polish with sanitation in DI H₂O after each round of polishing. Creation of the K⁺ selective membrane was performed by using the same ISE cocktail solution that was used with the PC substrates; 20 μl was pipetted onto the cleaned electrodes and allowed to dry for 24 hours. Planar carbon ISE's were conditioned in 10 mM KC1 for 24 hours prior to use and stored in the same solution.

A pseudo Ag/AgCl reference electrode was used for microfluidic experiments and was prepared by first exposing an Ag wire to a flame to clean the surface of the wire. The wire was then washed with ethanol and then placed in bleach until a chloride layer was formed, which was associated with a color change of the wire to a dark purple.

Microfluidic chip fabrication: Microfluidic chips were made in a layer-by-layer manner from PMMA (poly(methyl methacrylate)) sheets and Melinex® (a polyethylene terephthalate (PET) film) double-sided adhesive layers. A CO₂ laser was used to ablate patterns into the PMMA and the adhesive layers that were used in formation of the fluidic channels. Layers were assembled with a gig and pressed at 500 psi in order to remove air bubbles from the adhesive layers. O-rings were used between the PMMA layers in order to connect PEEK tubing for flow through experiments and for future vacuum connections involving interstitial fluid extraction. Ports were laser cut in the chip so that an Ag/AgCl wire and Pt counter could be used as a reference and counter electrode, respectively.

EMF Measurements: A CH Instruments multi-channel potentiostat was used to measure zero potential measurements of PC and PG/K+ISE membranes. In-solution measurements used an Ag/AgCl reference electrode and a Pt wire counter. A VoltaLab potentiostat was used for chronopotentiometric measurements. In these experiments, an Ag/AgCl reference and Pt wire counter electrode were used.

Microneedle fabrication: Hollow microneedles were prepared using a laser direct write system utilizing two-photon polymerization. First, a CAD file was created in the desired shape and dimensions of the microneedle and was uploaded to the LDW operating software (GOLD3D). The software sliced the CAD file and assigned laser and “writing” parameters such that the fabrication process can be optimized. The two photon polymerization effect is achieved with the help of Ti:Sapphire laser, which was operated at 800 nm, 150 fs, and 76 MHz. A 4× objective was used to focus the beam; write settings of 150 μm/s write speed, 2 μm x- and y-axis rastering, as well as 25 μm z-step height were used in this study. Eshell 300 (a liquid, photo-reactive acrylate) was used as the resin for both the hollow microneedles and the substrates.

Substrates were created in PDMS (poly(dimethylsiloxane)) molds made by laser cutting PMMA to 10 mm×10 mm×2 mm pieces and molding them with PDMS. Eshell 300 was then placed in the molds and cured with a UV lamp. In order to make a fluidic connection between the microfluidic chip and the hollow microneedle, a bore was cut into the substrates with a CO₂ laser such that the bore diameter was around 150 μm. Bore patterns were designed in CorelDraw and a single 100 μm circle (or array or circles) was drawn and uploaded to the laser cutting software. A well was made on top of the bore-containing substrate such that a microneedle could be written onto the substrate (FIG. 2A). The well was created with parafilm; once filled with resin, a glass cover slip was placed on top of the spacer. A vacuum was pulled from the bottom of the substrate such that a small amount of resin was pulled into the bore in order to remove air.

Discussion

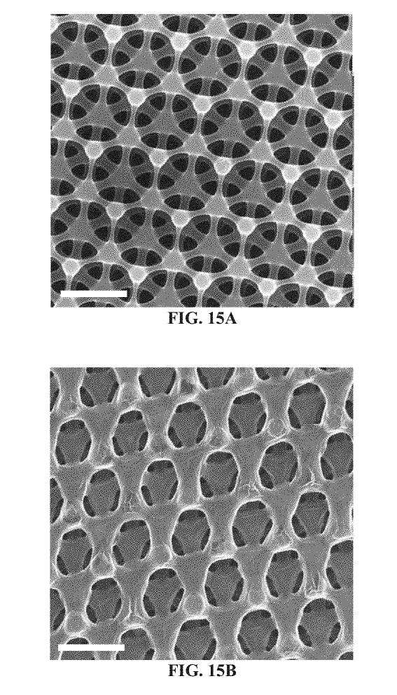

A scanning electron micrograph of the interferometric lithographically-fabricated pyrolyzed carbon (PC) electrode is shown in FIG. 15A. The electrodes resemble the colloidal crystal-templated 3DOM carbon prepared by Buhlman and Stein in appearance and have been previously shown to be similar in composition to an amorphous glassy carbon material (Lai C et al., Anal. Chem. 2007; 79:4621-6). The PC has a slightly more ordered architecture and fewer defects than the 3DOM, which should facilitate penetration of the ionophore-containing membrane. Therefore, we tested this novel ordered carbon electrode for its potential as a solid state ISE. We have shown that such a highly ordered periodic porous carbon structure produces a high surface area/high mass transport environment, which is suitable for electrochemical applications as for use as a scaffold for a variety of material modifications (Burckel D B et al., Small 2009; 5:2792-6).

The conversion of the PC to graphene (PG) occurred after nickel coating and annealing was performed (Xiao X et al., ACS Nano 2012; 6:3573-9). As can be seen in the SEM of the PG electrode (FIG. 15B), there is an increase in overall arm diameter after conversion, which indicates a concomitant decrease in pore size. The surface roughness also changes dramatically, transitioning from near atomic smoothness (Burckel D B et al., Small 2009; 5:2792-6) to a wrinkled graphene surface. After modification with the K⁺ membrane cocktail (described in the experimental section), reversed chronopotentiometric scans were performed to determine capacitance and stability of the polymeric membrane-modified electrodes in comparison to a planar glassy carbon electrode that was prepared in an identical manner (FIG. 16). The curves generated are a reflection of the resistance of the membrane and the ability of the membrane to adjust to changes in solution ion concentrations. The very small change in potential upon reverse biasing for the PC and PG electrodes versus the glassy carbon is indicative of increased stability. Resistance values of 12.75 and 12.69 MΩ were calculated for the PC and PG K+/ISE electrodes, respectively, for the response of the potential jump compared to 37.75 MΩ for the GCE. The increased stability is in accordance with the observations of 3DOM electrodes, where increased surface area and high capacitance provides a much more favorable environment for the integration of the polymeric membrane. These resistance numbers are similar to both carbon nanotubes and graphene solid state ISE transducers (Crespo G A et al., Anal. Chem. 2008; 80:1316-22; and Ping J. et al., Electrochem. Commun. 2011; 13:1529-32). The potential drift of the membranes was calculated to be 0.212 mV/s for the PC/K+ISE and 0.211 for the PG/K+ISE.

FIG. 17A-17B shows electromotor force (EMF) measurements of the PC and PG/K+ISE for increasing concentrations of potassium. Potentials in ISE measurements are related to the ionic activity of the ion being detected and the behavior of the potential as a function of the Nernst equation. For the PC/K+ISE, potential stabilization was rapid for each K⁺ spike (approximately 20 seconds, inset in FIG. 17A) and was comparable to other carbon-based transducers (Li F et al., Analyst 2012; 137:618-23). The potential response was near Nernstian (57.9 mV/decade), as seen in FIG. 17C, and the linear range was from 10⁻⁵ M to 10⁻² M with a detection limit of 10^{-5.65} M. The PG produced similar response times with a comparable linear range; however, large potential drifts were observed after each spike before a stable baseline was reached, indicating a much less stable electrode. Therefore, the PC electrodes were chosen for their superior stability over PG and used for integration into a microneedle ISE sensor.

FIG. 18 shows EMF measurements at the PC/K⁺1SE for alternating spikes of potassium and sodium in order to determine the influence of ion interference in a mixed solution. Normal physiological potassium levels are between 3 and 6 mM and normal sodium levels are between 135 and 145 mM; spikes were chosen to represent expected physiological fluctuations. Arrows in FIG. 18 shows alternating 5 mM KCl spikes and 10 mM NaCl spikes. Throughout this scan, the PC/K⁺SE rapidly responded to the KCl spikes; no influence was observed for additions of NaCl. These results indicate that the PC/K⁺ ISE was not significantly affected by the addition of a prevalent interfering ion and remained selective only to K⁺.

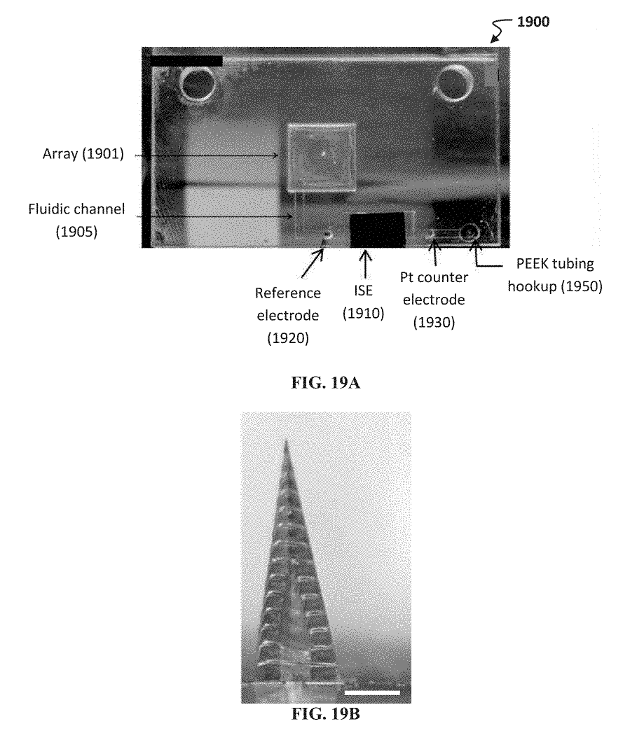

An advantage of using a lithographic approach for creation of a porous carbon ISE contact is the ability to photopattern the carbon electrode, which is useful for miniaturizing and integrating the electrode within a microfabricated device. FIG. 19A shows an optical image of an integrated microneedle with a PC/K+ISE in a microfluidic chip 1900. A fluidic channel 1905 (870 μm wide) and openings for placement of PC/K+ISE 1910 and microneedle array 1901 were cut into a 1.5 mm thick PMMA substrate via CO₂ laser machining A 8 mm×13 mm cut piece of lithographically patterned PC/K⁺ ISE was adhered into the channel reservoir with the double sided adhesive used for microfluidic chip assemble. A reference electrode 1920, a counter electrode 1930, and PEEK tubing 1950 were connected to the fluidic channel 1905. A plastic Melinex® adhesive plate was placed over the top to complete the channel.

Eshell 300, a Class 2a biocompatible material commonly used for hearing aid implants, was used to fabricate the microneedle (see, e.g., FIG. 19B); we previously demonstrated compatibility of this material with two-photon lithography as well as evaluated growth of human epidermal keratinocytes and human epidermal fibroblasts on this material (Gittard S D et al., Faraday Discuss. 2011; 149:171-85). Optimization of the laser energy to avoid resin burning/bubbling and over-polymerization (clogging) of the microneedle bore or sub-optimal laser output energies, which is associated with partial polymerization between the layers, is shown in FIG. 10A-10C. Based on these results, larger (>25 μm) step heights (write distance between layers) were obtained with the 4× objective. Overpolymerization of hollow microneedle bores was common with energies above 60 mW (measured at the stage); optimal operating energies for hollow microneedle fabrication around 50 mW were chosen to avoid laser power fluctuations. Thus, a 440 μm×1450 μm×165 μm microneedle (width, height, triangular bore) was fabricated and added into the microfluidic manifold.

Integration of the hollow microneedle with a microfluidic chip was achieved by writing a hollow microneedle onto a substrate which fit within a recess on the microfluidic chip (FIG. 2A-2D). Previous work by our group utilized 2PP to write both the substrate and the microneedle however this technique is time consuming and only the microneedle requires the fabrication resolution that 2PP offers (Gittard S D et al., Faraday Discuss. 2011; 149:171-85).

Initial experimental showed that writing Eshell 300 hollow microneedles directly onto the PMMA microfluidic chip created a weak bond; the microneedles tended to shear when placed into the skin. To circumvent this issue, we created the substrates from the same material as the microneedles so the bonds would be the same. Additionally, preformed substrates allowed for facile integration within the microfluidic chip.

To create the fluidic pathway from the microneedle to the microfluidics, a bore was created in the Eshell 300 substrate using a CO₂ laser. Laser cutting speeds were altered while parameters for power, z-height, resolution, and gas flow were maintained. Exit bores on the substrates were measured since this side of the bore had a smoother surface for writing needles and was smaller than the entrance bore due to thermal effects of the laser. Bore sizes needed to be smaller than the base of the microneedle but larger than the bore of the microneedle; a substrate bore of approximately 150 μm was used in this study. A range of laser cutting speeds was examined with 10 mm×10 mm×2 mm Eshell 300 substrate and was measured using a digital microscope. For this particular non-limiting example, the optimal laser cutting speed for producing exit bores in the Eshell 300 substrates were at 7 (arbitrary units).

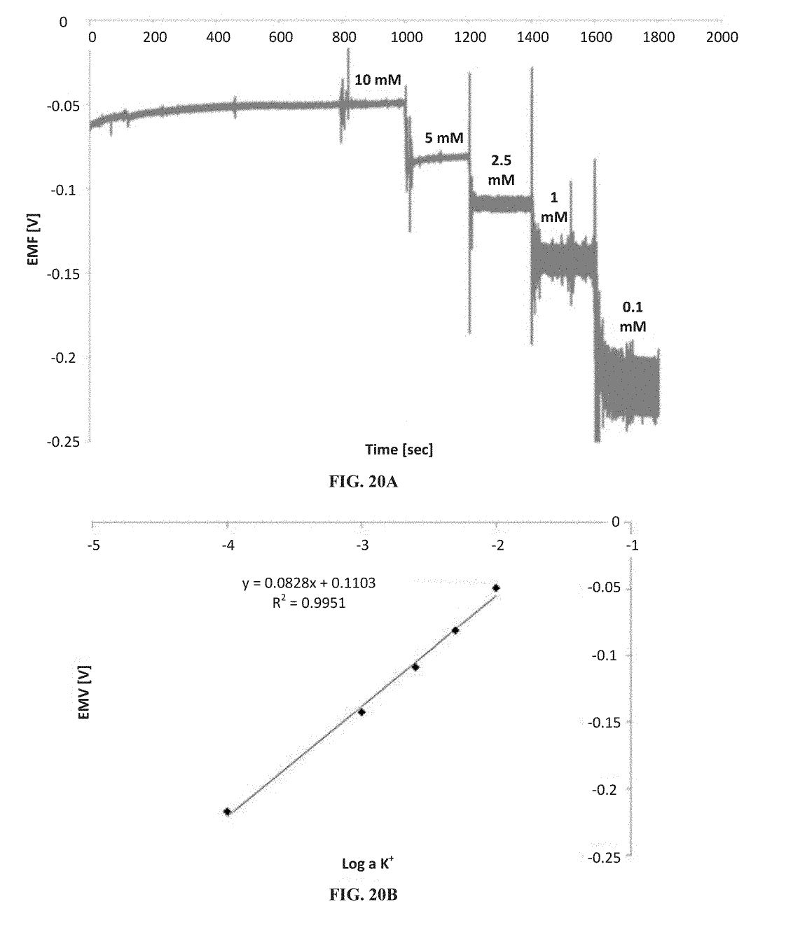

For on-chip measurements, KCl solutions were flowed through the chip and measured downstream at the PC/K+ISE shown in FIG. 20. Measurements from the PC/K⁺ ISE were obtained versus an Ag/AgCl wire reference and Pt counter electrode, respectively, that were also integrated into the fluidic channel. The inset in FIG. 20 shows a calibration curve generated from the K⁺ spikes introduced to the fluidic chip. A linear response was noticed for the tested values; however, the response was super Nernstian, which was attributed to the Ag/AgCl reference wire. Ag/AgCl reference wires can be susceptible to fluctuations in potential when varying concentrations of chloride are introduced into the sample due to dissociation of chloride ions on the surface of the electrode. Subtracting the influence of ionic dissociation due the Ag/AgCl wire from the measured values can be used to compensate and plot an ideal Nernstian response. The on chip response of the PC/K⁺ ISE through fluidic introduction of K⁺ through the microneedle fluidic channel was responsive to physiological potassium levels, indicating the chip is capable of rapidly and selectively measuring clinically relevant samples corresponding to normal and abnormal concentrations.

Conclusion

In conclusion, we created a transdermal sensing device designed to measure physiologically relevant concentrations of potassium. Porous carbon and porous graphene electrodes were tested as transducers for ISE's. While they both were capable of lowering the membrane resistant of the ISE's when compared to glassy carbon electrodes, the porous carbon electrodes showed better electrochemical performance. Porous carbon K⁺ ISE's exhibited a detection range from 10⁻⁵ M to 10⁻² M with a near Nernstian slope of 57.9 mV/decade and rapid stabilization concentration changes (˜20 s). Porous graphene K⁺ ISE's also exhibited rapid EMF changes to potassium spikes however stability of these electrodes was poor. Porous carbon K⁺ ISE's showed no EMF response to NaCl spikes which are known to cause ion interference and would be prevalent in real samples. A method to incorporate hollow microneedles made via two-photon polymerization into a microfluidic chip was described. The method allows for a hollow microneedle to draw fluid over a three-electrode system within a microfluidic chip which provides an attractive platform for an on-body sensing system for monitoring potassium.

### Example 3

### Minimally Invasive Electrolyte Monitoring of Ebola Patients

Electrolyte imbalance and dehydration are critical factors that contribute to mortality in patients with an Ebola virus infection. Current field protocols to measure electrolytes require blood draws that are undesirable due to the transfer of large volumes of fluids between patient and diagnostic components, waste requirements, and possibilities of accidental puncturing of personal protective equipment (PPE) from the use of syringes, all of which contribute to an increased chance of infection to the health worker and others.

We have developed a transdermal sensing platform based on microneedles. Microneedles are advantageous over traditional needles as their size enables minimally invasive interrogation due to their ability to puncture the skin's stratum corneum and access interstitial fluid without irritating deeper layers of the skin associated with pain, blood flow, or sensation. The platform employs microneedle-based sensors to monitor ascorbic acid, glucose, lactate, pH, and potassium (see, e.g., Miller P R et al., “Integrated carbon fiber electrodes within hollow polymer microneedles for transdermal electrochemical sensing,” Biomicrofluidics 2011; 5:013415 (14 pages); Miller P R et al., “Multiplexed microneedle-based biosensor array for characterization of metabolic acidosis,” Talanta 2012; 88:739-42; and Miller P R et al., “Microneedle-based transdermal sensor for on-chip potentiometric determination of K⁺,” Adv. Healthcare Mater. 2014; 3(6):876-81).

Here, we propose to develop a simple and disposable microneedle-based electrolyte sensing platform that obviates the need to draw large volumes of blood. The platform is syringe free, minimizes waste, and can easily be handled by healthcare workers wearing cumbersome PPE with zero possibility of accidental puncturing of PPE due to the small size of the microneedles. Key components of the platform include a one-shot, single use disposable microneedle cartridge; a detector configured to interface with the cartridge; and an electronic readout interface, which can be integrated with the detector or can be a stand-alone readout device (e.g., a smart phone) that wirelessly interact with the detector (e.g., by way of Bluetooth wireless technology).

In particular, the disposable microneedle cartridge can be attached and discarded with a simple “lock and release” mechanism and requires only tens of microliters of interstitial fluid for measurements, thereby minimizing waste. This mechanism allows the cartridge to be mounted on the handheld detector and discarded with minimal effort and contact with the user (e.g., the healthcare professional).

In use, the sample from the patient is accessed by placing the microneedle array at the test site. The desired sample (e.g., interstitial fluid and/or blood) then flows into the bore(s) of the microneedle(s) and is delivered to the sensing portion (e.g., one or more transducers or electrodes) of the detector for real-time detection (see, e.g., FIG. 22). Alternatively, the desired sample is stored for analysis at a later time (see, e.g., FIG. 23). The sample fluid can enter a chamber of the cartridge through passive diffusion or active diffusion (e.g., by using an active pumping mechanism built into the body section). For real-time analysis, the detector can contain an internal ion selective electrode transducer for electrolytes (such as potassium or sodium) contained within the body of the detector. For sample acquisition, the detector can include a pumping mechanism or a vacuum source within the body, which can assist in drawing the fluid sample through the microneedle and into a chamber within the cartridge.

### Other Embodiments

All publications, patents, and patent applications mentioned in this specification, including U.S. Provisional Application No. 61/902,617, filed Nov. 11, 2013, are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A multilayered device for detecting one or more markers in a sample comprising:
an array comprising a plurality of hollow needles and a substrate coupled to the plurality of hollow needles, wherein each of the plurality of hollow needles has an interior surface facing a hollow lumen and an exterior surface, a distal end of the exterior surface for at least one needle comprises a puncturing edge, and at least one of the plurality of hollow needles has a length of more than about 0.5 mm; and wherein the substrate comprises an inlet in fluidic communication with a proximal end of at least one of the plurality of hollow needles;
a first layer comprising an aperture configured to include the array;
a second layer comprising a first chamber configured to be in fluidic communication with the inlet of the substrate and a first channel in fluidic communication with the first chamber, wherein the second layer is disposed below the first layer; and
a third layer comprising a port configured to place a sensing transducer in fluidic communication with the first channel, wherein the sensing transducer is configured to detect said one or more markers in the sample and wherein the third layer is disposed below the second layer.

2. The device of claim 1, further comprising a mixing chamber in fluidic communication with the first channel.

3. The device of claim 2, further comprising a reaction chamber in fluidic communication with the mixing chamber.

4. The device of claim 2, further comprising a reagent chamber in fluidic communication with the mixing chamber.

5. The device of claim 1, wherein the plurality of hollow needles is configured to obtain the sample from a subject.

6. The device of claim 5, wherein the puncturing edge of one or more of the plurality of hollow needles comprises a tapered point, a sharpened bevel, or one or more prongs.

7. The device of claim 1, wherein at least one of the plurality of hollow needles comprises a polymer, a metal, silicon, glass, or a composite material.

8. The device of claim 1, wherein the sensing transducer is selected from the group consisting of an electrode, an ion selective electrode, and an optical sensor.

9. The device of claim 8, wherein the sensing transducer is an ion selective electrode comprising a porous material and one or more ionophores.

10. The device of claim 8, wherein the sensing transducer further comprises a modified surface.

11. The device of claim 10, wherein the sensing transducer comprises an array of electrodes.

12. The device of claim 8, wherein the sensing transducer is an electrode selected from the group of a planar electrode, a three-dimensional electrode, a porous electrode, a microelectrode, and a nanoelectrode.

13.-15. (canceled)

16. The device of claim 1, wherein the plurality of hollow needles and the substrate are configured as a disposable cartridge module.

17. The device of claim 16, wherein the disposable cartridge module comprises:
a barrel comprising an internal volume, a distal end, and a proximal end, wherein the distal end of the barrel is coupled to the plurality of hollow needles and the substrate and the proximal end of the barrel comprises an opening.

18.-19. (canceled)

20. A kit comprising:
a device of claim 1; and
(ii) instructions for affixing the device to a subject and activating the device.

21. The kit of claim 20, further comprising a therapeutic agent selected from the group consisting of an anesthetic, an antiseptic, an anticoagulant, a drug, and a vaccine.

22.-29. (canceled)

30. A platform comprising a disposable cartridge module and a handheld module,
wherein the disposable cartridge module comprises:
an array comprising a plurality of hollow needles and a substrate coupled to the plurality of hollow needles, wherein each of the plurality of hollow needles has an interior surface facing a hollow lumen and an exterior surface, a distal end of the exterior surface for at least one needle comprises a puncturing edge, and at least one needle has a length of more than about 0.5 mm; and wherein the substrate comprises an inlet in fluidic communication with a proximal end of at least one of the plurality of hollow needles;
a first layer comprising an aperture configured to include the array;
a barrel comprising an internal volume, a surface portion defining the internal volume, a distal end, and a proximal end, wherein the distal end of the barrel is coupled to the plurality of hollow needles and the substrate, or a portion thereof, and the proximal end of the barrel comprises an opening; and
a ridge disposed on the surface portion of the barrel; and
wherein the handheld module comprises:
a body, wherein the body comprises a distal section and a proximal section;
a central bore disposed within the body and in fluidic communication with the disposable cartridge module;
a mounting shaft disposed on the distal section of the body, wherein the mounting shaft is configured to be inserted into the opening of the disposable cartridge module; and
a stop member structure disposed on an outer surface portion of the mounting shaft, wherein the stop member structure is configured to interface with the ridge of the disposable cartridge module.

31. The platform of claim 30, wherein the body further comprises a sensing transducer in fluidic communication with the internal volume, and wherein the sensing transducer is configured to detect one or more markers in the sample.

32. The platform of claim 30, wherein the body further comprises a pump configured to transport the sample from the plurality of hollow needles and/or the internal volume into the central bore.

33. The platform of claim 30, wherein the handheld module further comprises an electronic readout interface configured to wirelessly communicate with the handheld module, and wherein the electronic readout interface is selected from the group consisting of a smartphone, a cell phone, a mobile device, or a mobile phone.

34. The platform of claim 30, further comprising a chamber in fluidic communication with the disposable sample module and/or the central bore of the handheld module.

35.-37. (canceled)

38. The device of claim 1, further comprising:
a fourth layer comprising a plurality of accesses in fluidic communication with the port, wherein the fourth layer is disposed below the third layer.

39. The device of claim 38, further comprising:
a base layer comprising a plurality of further accesses in fluidic communication with the plurality of accesses in the fourth layer, wherein the base layer is disposed below the fourth layer.

40. The device of claim 39, further comprising one or more wiring, electrodes, or fluidic connections inserted into at least one of the plurality of further accesses in the base layer.

41. A platform comprising:
a disposable cartridge comprising the multilayered device of claim 1; and
a handheld module comprising a body and a central bore configured to be in fluidic communication with the disposable cartridge.

42. The platform of claim 41, wherein a distal portion of the body comprises a mounting shaft configured to interface with the disposable cartridge, and wherein a proximal portion of the body includes a handle.

43. The platform of claim 42, wherein body further comprises a release lever configured to detach the disposable cartridge from the handheld module.

44. The platform of claim 42, wherein the body is configured to interface with an electronic readout interface, and wherein the electronic readout interface is selected from the group consisting of a smartphone, a cell phone, a mobile device, or a mobile phone.

45. The platform of claim 30, wherein the disposable cartridge module further comprises an o-ring disposed on the surface portion of the barrel.

46. The device of claim 1, further comprising a reaction chamber in fluidic communication with the first channel.

47. The device of claim 46, further comprising a mixing chamber in fluidic communication with the reaction chamber.

48. The device of claim 46, further comprising a reagent chamber in fluidic communication with the reaction chamber.

49. The device of claim 9, wherein the porous material comprises porous carbon, graphene, silicon, or conducting polymer.

50. The device of claim 11, wherein the array of electrodes comprises immobilized capture antibodies.
